(19) United States
(12) Patent Application Publication
(10) Pub. No.: US 2026/0152496 A1
(43) Pub. Date: Jun. 4, 2026

(54) IMPROVED PROCESSES FOR PREPARING DIMETHYL-2,3-DIHYDRO-1H-INDENE DERIVATIVES

(71) Applicants: YUHAN CORPORATION, Seoul (KR); GREEN CROSS CORPORATION, Yongin-si, Gyeonggi-do (KR)

(72) Inventors: Jun-Sup LEE, Yongin-si, Gyeonggi-do (KR); Jong-Chul LIM, Yongin-si, Gyeonggi-do (KR); Jung-Ae LEE, Seoul (KR); Hyun JU, Hwaseong-si, Gyeonggi-do (KR); Woo-Seob SHIN, Suwon-si, Gyeonggi-do (KR); Kyung-Min KO, Yongin-si, Gyeonggi-do (KR); Yoo-Jin SIM, Yongin-si, Gyeonggi-do (KR); Yoon-Ah HWANG, Suwon-si, Gyeonggi-do (KR); Se-Yong KIM, Yongin-si, Gyeonggi-do (KR); Hyun-Sik YUN, Yongin-si, Gyeonggi-do (KR); Sang-Ik SHIN, Yongin-si, Gyeonggi-do (KR)

(73) Assignees: YUHAN CORPORATION, Seoul (KR); GREEN CROSS CORPORATION, Yongin-si, Gyeonggi-do (KR)

(21) Appl. No.: 19/123,279

(22) PCT Filed: Oct. 23, 2023
(86) PCT No.: PCT/KR2023/016442

§ 371 Date: Apr. 22, 2025

(30) Foreign Application Priority Data
Oct. 24, 2022 (KR) 10-2022-0137641
Jun. 2, 2023 (KR) 10-2023-0071386

(51) Int. Cl. C07D 453/02; C07C 209/68; C07C 211/42; C07C 217/74

(52) U.S. Cl. CPC C07D 453/02; C07C 209/68; C07C 211/42; C07C 217/74; C07C 2531/16; C07C 2602/08

(57) ABSTRACT

Provided are an improved process for preparing a dimethyl-2,3-dihydro-1H-indene derivative of Formula 1 or a pharmaceutically acceptable salt thereof, and intermediates useful for the process, where the process for preparing a compound of Formula 1 or its stereoisomer via the intermediate(s) allows to minimize the amount of heavy metal (Pd) remaining in the final product, i.e., the compound of Formula 1 or its stereoisomer, and to prepare a stereoisomer of the compound of Formula 1 or a pharmaceutically acceptable salt thereof in high optical purity.

### TECHNICAL FIELD

The present invention relates to an improved process for preparing a dimethyl-2,3-dihydro-1H-indene derivative or a pharmaceutically acceptable salt thereof. In addition, the present invention relates to novel intermediates useful in the process.

### BACKGROUND ART

Dimethyl-2,3-dihydro-1H-indene derivatives or pharmaceutically acceptable salts thereof, including the compound of Formula 1 below, are known as compounds having excellent inhibitory activity against glucosylceramide synthase (GCS) (WO 2021/096238).

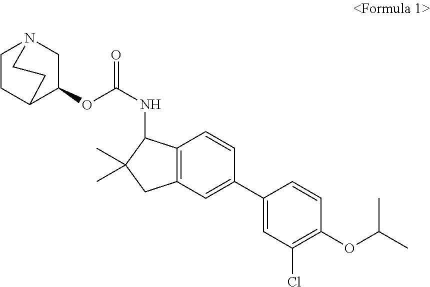 [SMILES: CC(C)Oc1ccc(-c2ccc3c(c2)CC(C)(C)C3NC(=O)O[C@@H]2CN3CCC2CC3)cc1Cl]

WO 2021/096238 discloses a process for preparing a dimethyl-2,3-dihydro-1H-indene derivative, for example, the process as shown in Reaction Scheme 1 below.

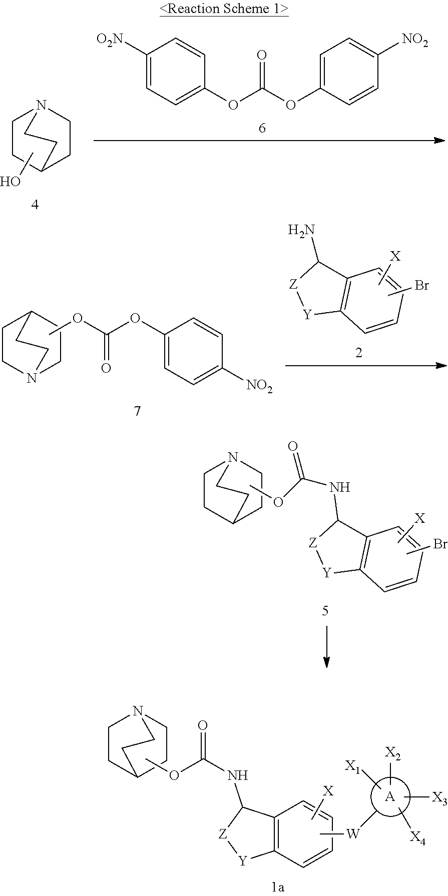

The process according to Reaction Scheme 1 comprises preparing a product (e.g., the compound of Formula 1 in the form of free base) through a Suzuki coupling reaction between a compound of Formula 5 (e.g., (S)-quinuclidin-3-yl (5-bromo-2,2-dimethyl-2,3-dihydro-1H-inden-1-yl) carbamate) and an A-W-boronic acid substituted with X₁, X₂, X₃, or X₄ (e.g., 3-chloro-4-isopropoxyphenylboronic acid) in the final step. The Suzuki coupling reaction involves the use of a palladium catalyst such as [1,1′-bis(diphenylphosphino)ferrocene]dichloropalladium(II) (Pd(dppf)Cl₂). However, the Suzuki coupling reaction carried out in the final step causes a high risk that the heavy metal such as palladium, the ligand used for carrying out the Suzuki coupling reaction, and the by-products therefrom remain in the product (e.g., the compound of Formula 1 in the form of free base).

### DISCLOSURE OF INVENTION

### Technical Problem

The present inventors carried out various studies to develop an improved process for preparing the compound of Formula 1 or a pharmaceutically acceptable salt thereof. The present inventors have found that, when the compound of Formula 1 or its stereoisomer is prepared via novel intermediate(s), it is possible to minimize the amount of heavy metal (Pd) and the by-products remaining in the final product (i.e., the compound of Formula 1 or its stereoisomer).

In addition, the present inventors have developed a process capable of converting one of the intermediates (i.e., 5-bromo-2,2-dimethyl-2,3-dihydro-1H-inden-1-amine) to (R)-5-bromo-2,2-dimethyl-2,3-dihydro-1H-inden-1-amine in high optical purity. And, the present inventors have found that, when the compound of Formula 1 is prepared by using the obtained (R)-5-bromo-2,2-dimethyl-2,3-dihydro-1H-inden-1-amine and the intermediate(s), it is possible to prepare a stereoisomer of the compound of Formula 1 in high optical purity.

Therefore, the present invention provides an improved process for preparing a compound of Formula 1 or a pharmaceutically acceptable salt thereof, comprising the use of the novel intermediates.

And, the present invention provides the novel intermediates.

In addition, the present invention provides a process for preparing (R)-5-bromo-2,2-dimethyl-2,3-dihydro-1H-inden-1-amine having high optical purity.

### Solution to Problem

According to an aspect of the present invention, there is provided a process for preparing a compound of Formula 1 or a pharmaceutically acceptable salt thereof, the process comprising:
(a) reacting a compound of Formula 4 with 3-chloro-4-isopropoxyphenylboronic acid, followed by reacting with D-tartaric acid, to prepare a compound of Formula 3;
(b) reacting the compound of Formula 3 with a base to prepare a compound of Formula 2; and
(c) reacting the compound of Formula 2 with (S)-4-nitrophenyl quinuclidin-3-yl carbonate to prepare a compound of Formula 1.

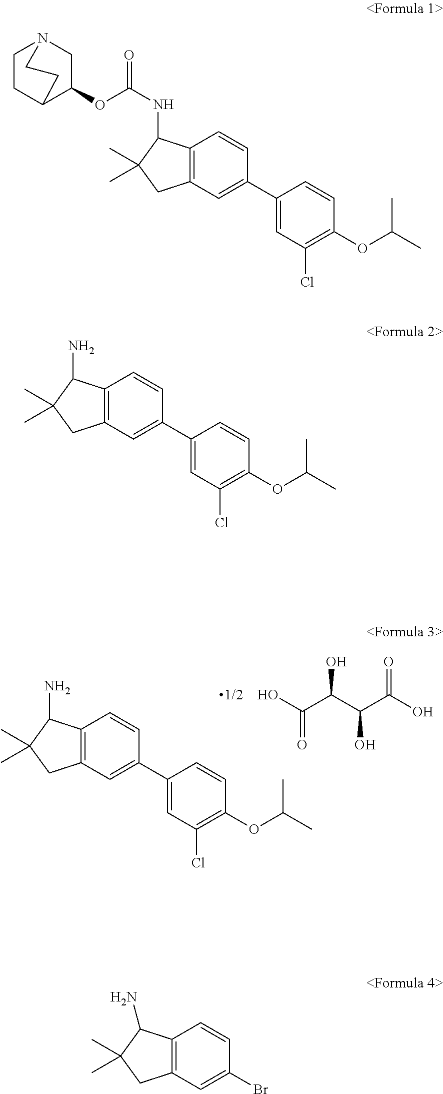

According to another aspect of the present invention, there is provided a process for preparing a compound of Formula 1a or a pharmaceutically acceptable salt thereof, the process comprising:
(a′) reacting a compound of Formula 4a with 3-chloro-4-isopropoxyphenylboronic acid, followed by reacting with D-tartaric acid, to prepare a compound of Formula 3a;
(b′) reacting the compound of Formula 3a with a base to prepare a compound of Formula 2a; and
(c′) reacting the compound of Formula 2a with (S)-4-nitrophenyl quinuclidin-3-yl carbonate to prepare a compound of Formula 1a.

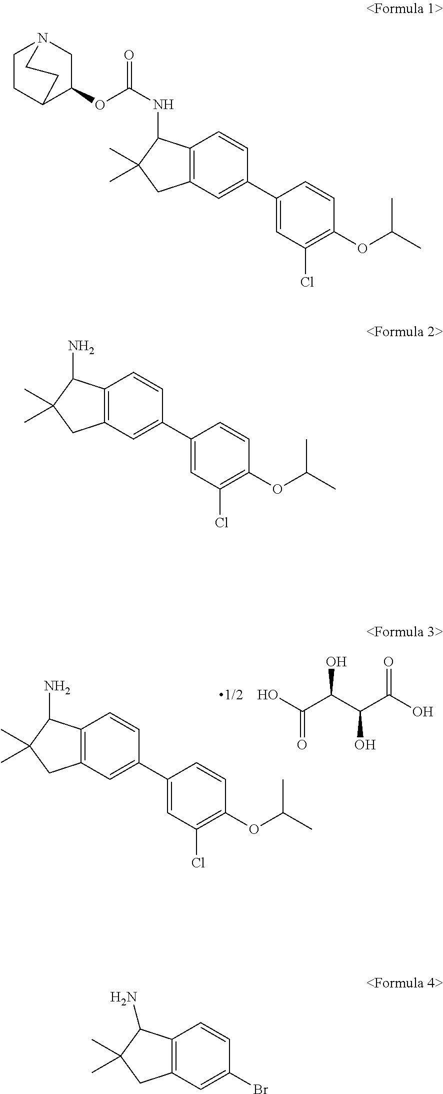

According to still another aspect of the present invention, there is provided a process for preparing a compound of Formula 4a comprising:
(i) reacting a compound of Formula 4 with N-acetyl-D-leucine, in a water-miscible organic solvent or a mixed solvent of water and a water-miscible organic solvent, to obtain a compound of Formula 5; and

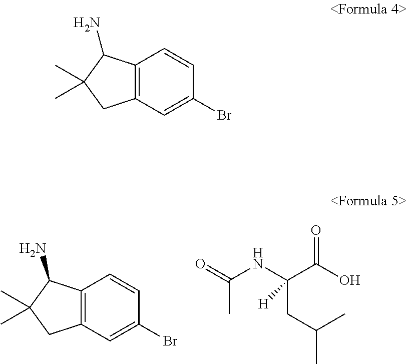

(ii) reacting the compound of Formula 5 with a base to obtain a compound of Formula 4a.

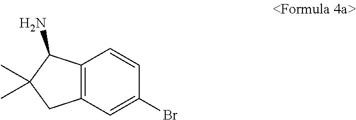

According to still another aspect of the present invention, there is provided a process for preparing a compound of Formula 4a comprising:
(p) reacting a compound of Formula 9 with iodomethane to prepare a compound of Formula 8;

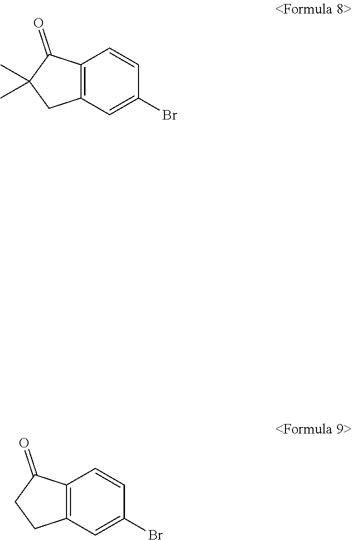

(q) reacting the compound of Formula 8 with (S)-(−)-methyl-2-propanesulfinamide to prepare a compound of Formula 7;

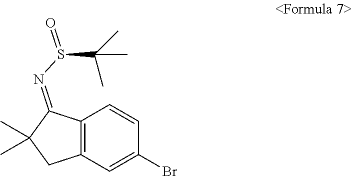 [SMILES: CC1(C)Cc2cc(Br)ccc2/C1=N\[S@@](=O)C(C)(C)C]

(r) reducing the compound of Formula 7 to prepare a mixture of a compound of Formula 6a and a compound of Formula 6b;

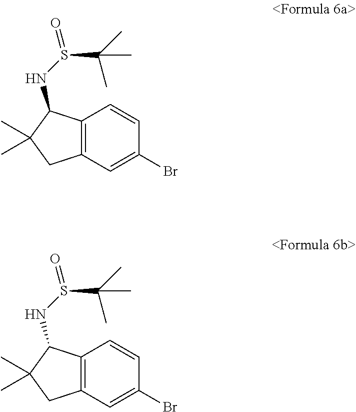 [SMILES: CC1(C)Cc2cc(Br)ccc2[C@@H]1N[S@@](=O)C(C)(C)C ; CC1(C)Cc2cc(Br)ccc2[C@H]1N[S@@](=O)C(C)(C)C]

(s) reacting the mixture of a compound of Formula 6a and a compound of Formula 6b with an acid to prepare a mixture of a compound of Formula 4a and a compound of Formula 4b, reacting the mixture of a compound of Formula 4a and a compound of Formula 4b with N-acetyl-D-leucine in a water-miscible organic solvent or a mixed solvent of water and water-miscible organic solvent, and then isolating a compound of Formula 5; and

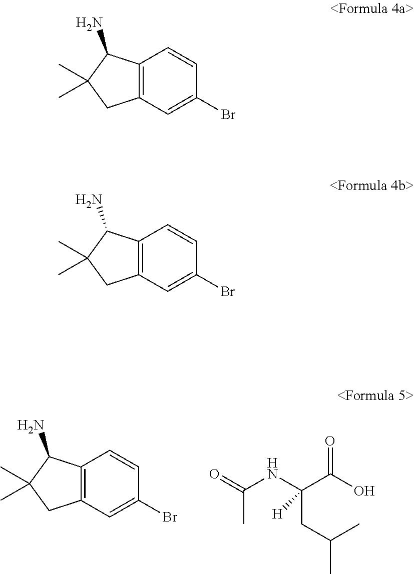

(t) reacting the compound of Formula 5 with a base to obtain a compound of Formula 4a.

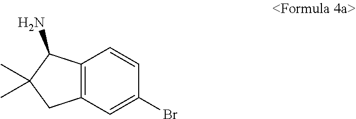

According to still another aspect of the present invention, there is provided an intermediate useful for the preparation of the compound of Formula 1, i.e. a compound of Formula 2:

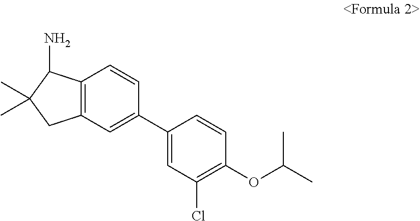 [SMILES: CC(C)Oc1ccc(-c2ccc3c(c2)CC(C)(C)C3N)cc1Cl]

According to still another aspect of the present invention, there is provided an intermediate useful for the preparation of the compound of Formula 1a, i.e. a compound of Formula 2a:

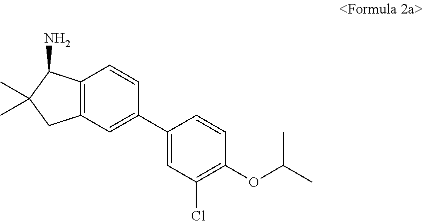 [SMILES: CC(C)Oc1ccc(-c2ccc3c(c2)CC(C)(C)[C@H]3N)cc1Cl]

According to still another aspect of the present invention, there is provided an intermediate useful for the preparation of the compound of Formula 1, i.e. a compound of Formula 3:

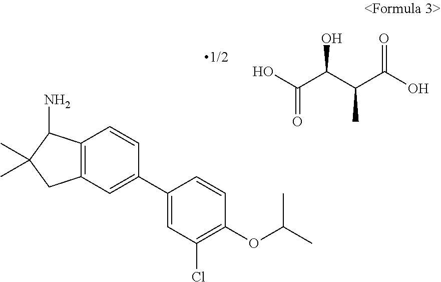

According to still another aspect of the present invention, there is provided an intermediate useful for the preparation of the compound of Formula 1a, i.e. a compound of Formula 3a:

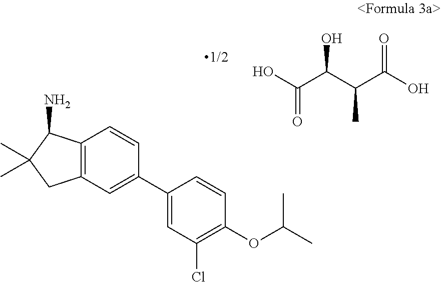

According to still another aspect of the present invention, there is provided an intermediate useful for the preparation of the compound of Formula 1a, i.e. a compound of Formula 5:

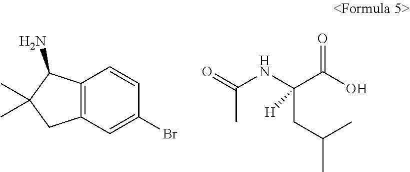

### Advantageous Effects of Invention

When the compound of Formula 1 or its stereoisomer is prepared via novel intermediate(s) according to the present invention, it is possible to minimize the amount of heavy metal (Pd) remaining in the final product (i.e., the compound of Formula 1 or its stereoisomer). In addition, when the compound of Formula 1 is prepared by converting 5-bromo-2,2-dimethyl-2,3-dihydro-1H-inden-1-amine to (R)-5-bromo-2,2-dimethyl-2,3-dihydro-1H-inden-1-amine; and then preparing the compound of Formula 1a via said novel intermediate(s) according to the present invention, it is possible to prepare a stereoisomer of the compound of Formula 1 or a pharmaceutically acceptable salt thereof in high optical purity.

### BEST MODE FOR CARRYING OUT THE INVENTION

The present invention provides an improved process for preparing (S)-quinuclidin-3-yl (5-(3-chloro-4-isopropoxyphenyl)-2,2-dimethyl-2,3-dihydro-1H-inden-1-yl) carbamate, i.e., the compound of Formula 1 or a pharmaceutically acceptable salt thereof. That is, the present invention provides a process for preparing a compound of Formula 1 or a pharmaceutically acceptable salt thereof, the process comprising:
(a) reacting a compound of Formula 4 with 3-chloro-4-isopropoxyphenylboronic acid, followed by reacting with D-tartaric acid, to prepare a compound of Formula 3;
(b) reacting the compound of Formula 3 with a base to prepare a compound of Formula 2; and
(c) reacting the compound of Formula 2 with (S)-4-nitrophenyl quinuclidin-3-yl carbonate to prepare a compound of Formula 1.

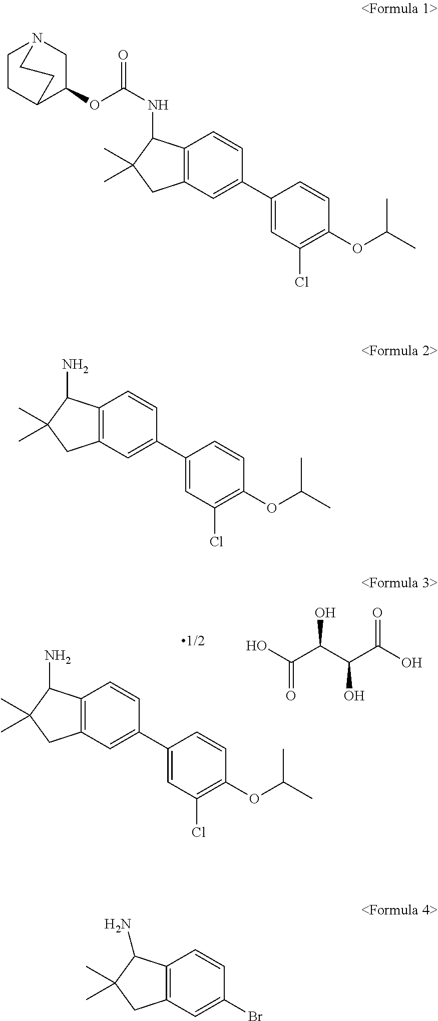

The present invention also provides an improved process for preparing a stereoisomer of the compound of Formula 1, i.e., (S)-quinuclidin-3-yl ((R)-5-(3-chloro-4-isopropoxyphenyl)-2,2-dimethyl-2,3-dihydro-1H-inden-1-yl) carba mate or a pharmaceutically acceptable salt thereof. That is, the present invention provides a process for preparing a compound of Formula 1a or a pharmaceutically acceptable salt thereof, the process comprising:
(a′) reacting a compound of Formula 4a with 3-chloro-4-isopropoxyphenylboronic acid, followed by reacting with D-tartaric acid, to prepare a compound of Formula 3a;
(b′) reacting the compound of Formula 3a with a base to prepare a compound of Formula 2a; and
(c′) reacting the compound of Formula 2a with (S)-4-nitrophenyl quinuclidin-3-yl carbonate to prepare a compound of Formula 1a.

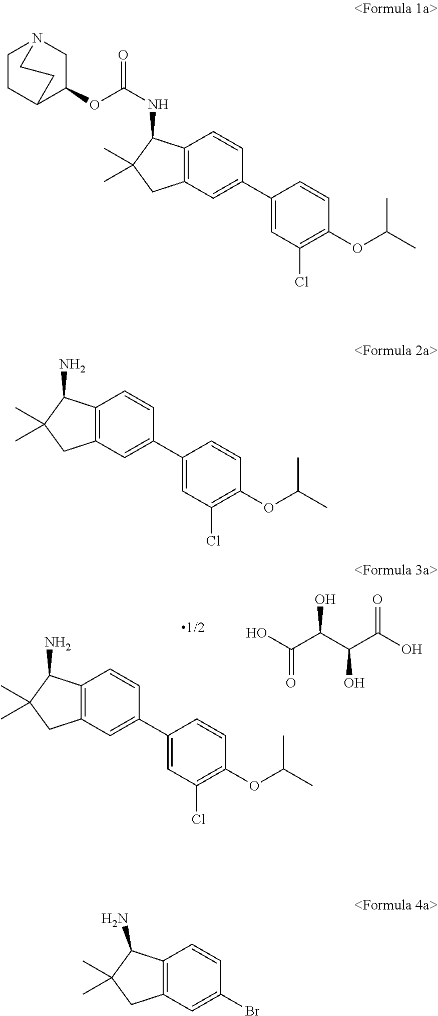

In the processes of the present invention, the compound of Formula 4 (i.e., 5-bromo-2,2-dimethyl-2,3-dihydro-1H-inden-1-amine) is a known compound and thus may be purchased commercially or synthesized according to the literature.

It been found by the present invention that, when the compound of Formula 4 is resolved by using a specific optical resolving agent (i.e., N-acetyl-D-leucine), it is possible to prepare (R)-5-bromo-2,2-dimethyl-2,3-dihydro-1H-inden-1-amine in high optical purity.

In an embodiment, the compound of Formula 4a may be prepared by a process comprising:
(i) reacting a compound of Formula 4 with N-acetyl-D-leucine, in a water-miscible organic solvent or a mixed solvent of water and a water-miscible organic solvent, to obtain a compound of Formula 5; and

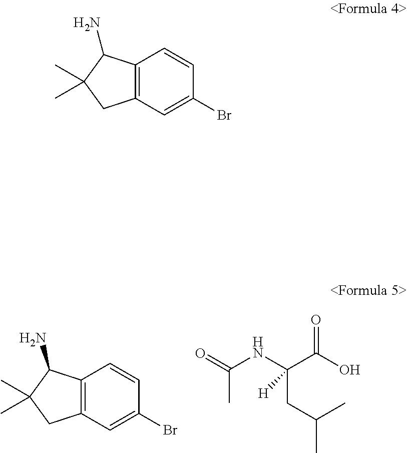

(ii) reacting the compound of Formula 5 with a base to obtain a compound of Formula 4a.

In Step (i), the resolving agent, i.e., N-acetyl-D-leucine, may be used in a ratio of 0.3 to 10 equivalents based on 1 equivalent of the compound of Formula 4, but is not limited thereto. The water-miscible organic solvent may be one or more selected from the group consisting of acetone, acetonitrile, C₁˜C₅ alcohol, tetrahydrofuran, methyl ethyl ketone, isopropyl acetate, ethyl acetate, toluene, methyl tert-butyl ether, 2-methyltetrahydrofuran, and dichloromethane, preferably acetone or acetonitrile. The volume ratio of the water-miscible organic solvent and water in the mixed solvent may be in the range of 3:1 to 20:1, for example, about 9:1. The reaction of Step (i) may be carried out at a temperature of 0° C. to 70° C. (e.g., about 35° C.) for 1 to 24 hours. The solid produced in Step (i), i.e., (R)-5-bromo-2,2-dimethyl-2,3-dihydro-1H-inden-1-amine acetyl-D-leucinate of Formula 5, may be isolated according to conventional methods such as filtering and drying (e.g., drying in vacuo).

In Step (ii), the base may be one or more selected from the group consisting of sodium hydroxide, potassium hydroxide, potassium carbonate, sodium carbonate, calcium hydroxide, sodium bicarbonate, and potassium phosphate; for example, sodium hydroxide may be used. The base may be used in a ratio of 1 to 3 equivalents based on 1 equivalent of the compound of Formula 5, but is not limited thereto. The reaction of Step (ii) may be carried out in a mixed solvent of water and an organic solvent such as isopropyl acetate, ethyl acetate, toluene, methyl tert-butyl ether, 2-methyltetrahydrofuran, dichloromethane, and the like. The volume ratio of the organic solvent and water in the mixed solvent may be in the range of 1:1 to 10:1, but is not limited thereto. The product, i.e., (R)-5-bromo-2,2-dimethyl-2,3-dihydro-1H-inden-1-amine, may be isolated according to conventional methods such as washing, drying, concentrating, and the like.

In another embodiment, the compound of Formula 4a may be prepared by a process comprising:
(p) reacting a compound of Formula 9 with iodomethane to prepare a compound of Formula 8;

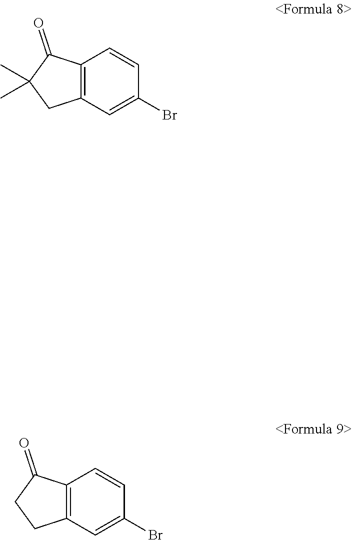

(q) reacting the compound of Formula 8 with (S)-(−)-methyl-2-propanesulfinamide to prepare a compound of Formula 7;

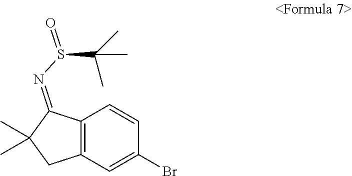 [SMILES: CC1(C)Cc2cc(Br)ccc2/C1=N\[S@@](=O)C(C)(C)C]

(r) reducing the compound of Formula 7 to prepare a mixture of a compound of Formula 6a and a compound of Formula 6b;

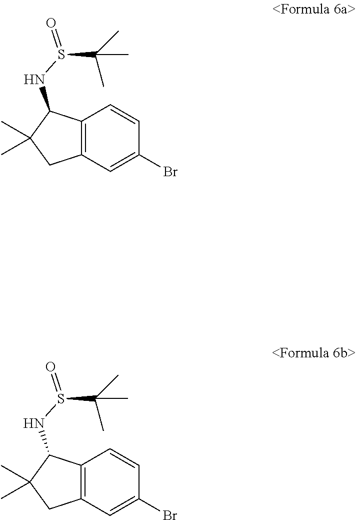 [SMILES: CC1(C)Cc2cc(Br)ccc2[C@@H]1N[S@@](=O)C(C)(C)C ; CC1(C)Cc2cc(Br)ccc2[C@H]1N[S@@](=O)C(C)(C)C]

(s) reacting the mixture of a compound of Formula 6a and a compound of Formula 6b with an acid to prepare a mixture of a compound of Formula 4a and a compound of Formula 4b, reacting the mixture of a compound of Formula 4a and a compound of Formula 4b with N-acetyl-D-leucine in a water-miscible organic solvent or a mixed solvent of water and water-miscible organic solvent, and then isolating a compound of Formula 5; and

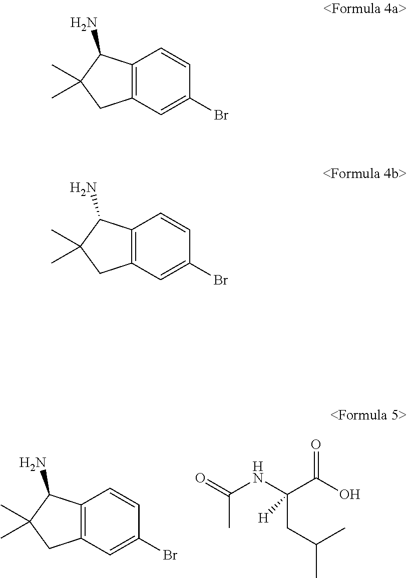

(t) reacting the compound of Formula 5 with a base to obtain a compound of Formula 4a.

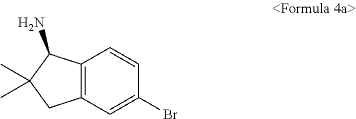

In Step (p), iodomethane may be used in a ratio of 2.0 to 3.0 equivalents based on 1 equivalent of the compound of Formula 9 (5-bromo-2,3-dihydro-1H-inden-1-one), but is not limited thereto. The reaction of Step (p) may be carried out in the presence of a base in a water-miscible organic solvent or a mixed solvent of water and a water-miscible organic solvent. The water-miscible organic solvent may be one or more selected from the group consisting of N-methyl-2-pyrrolidone, acetonitrile, tetrahydrofuran, and dimethylformamide. The volume ratio of the water-miscible organic solvent and water in the mixed solvent may be in the range of 1:1 to 10:1, for example, about 6:1. The base may be selected from the group consisting of cesium carbonate (Cs₂CO₃), sodium carbonate (Na₂CO₃), potassium carbonate (K₂CO₃), potassium phosphate (K₃PO₄), triethylamine, diisopropylamine, potassium hydroxide, potassium acetate, potassium t-butoxide, sodium hydroxide, sodium hydride, sodium phosphate, 1,8-diazabicyclo[5.4.0]undec-7-ene, 1,4-diazabicyclo[2.2.2]octane, 1,5-diazabicyclo[4.3.0]non-5-ene, pyridine, and diisopropylethylamine. The base may be used in a ratio of 2.0 to 3.0 equivalents based on 1 equivalent of the compound of Formula 9, but is not limited thereto. The reaction of Step (p) may be carried out at a temperature of 0° C. to 40° C. for 1 to 20 hours. The product of step (p), i.e., 5-bromo-2,2-dimethyl-2,3-dihydro-1H-inden-1-one of Formula 8, may be isolated according to conventional methods such as extracting, filtering, concentrating, and the like.

In Step (q), (S)-(−)-methyl-2-propanesulfinamide may be used in a ratio of 1.0 to 2.0 equivalents based on 1 equivalent of the compound of Formula 8, but is not limited thereto. The reaction of step (q) may be preferably carried out in the presence of a titanium catalyst such as titanium ethoxide, titanium isopropoxide and the like. The titanium catalyst may be used in a ratio of 2.0 to 3.0 equivalents based on 1 equivalent of the compound of Formula 8, but is not limited thereto. The reaction of Step (q) may be carried out in the presence of a solvent or in the absence of a solvent. When the reaction is carried out in the presence of a solvent, the solvent may be one or more selected from the group consisting of toluene, C₁˜C₁₀ alcohol, dichloromethane, tetrahydrofuran, acetonitrile, isopropyl acetate, dimethylformamide, dimethylacetamide, 2-methyltetrahydrofuran, ethyl acetate, and 1,4-dioxane. It has been found by the present invention that the reaction of Step (q) may be particularly preferably carried out under a nitrogen atmosphere at high temperature. Accordingly, the reaction of Step (q) may be preferably carried out at a temperature of 70° C. to 100° C., under a nitrogen atmosphere (e.g., while purging nitrogen gas). The product of Step (q), i.e., (S,Z)—N-(5-bromo-2,2-dimethyl-2,3-dihydro-1H-inden-1-ylidene)-2-methylpropane-2-sulfinamide of Formula 7, may be isolated according to a conventional method such as concentration; or be used in the subsequent reactions without isolating the residue obtained by the concentration.

In Step (r), the reduction may be carried out with a reducing agent. The reducing agent may be one or more selected from the group consisting of sodium triacetoxyborohydride, sodium cyanoborohydride, and sodium borohydride. The reducing agent may be used in a ratio of 1.0 to 2.0 equivalents based on 1 equivalent of the compound of Formula 7, but is not limited thereto. The reaction of Step (r) may be carried out at a temperature of 0° C. to 50° C. in an organic solvent such as tetrahydrofuran, toluene, C₁˜C₁₀ alcohol, dichloromethane, tetrahydrofuran, acetonitrile, dimethylformamide, dimethylacetamide, 2-methyltetrahydrofuran, 1,4-dioxane, and the like. The product of Step (r), i.e., a mixture of (S)—N—((R)-5-bromo-2,2-dimethyl-2,3-dihydro-1H-inden-1-yl)-2-methylpropane-2-sulfinamide of Formula 6a and (S)—N—((S)-5-bromo-2,2-dimethyl-2,3-dihydro-1H-inden-1-yl)-2-methylpropane-2-sulfinamide of Formula 6b, may be used in the subsequent reactions without isolating the residue obtained according to conventional methods such as extracting, concentrating, and the like.

In Step(s), the acid may be an inorganic acid such as hydrochloric acid, sulfuric acid, nitric acid, and the like. The acid may be used in a ratio of 2.0 to 10.0 equivalents based on 1 equivalent of the compound of Formula 6. The reaction between the mixture of the compound of Formula 6a and the compound of Formula 6b and the acid may be carried out at a temperature of 0° C. to 50° C. in an organic solvent such as ethyl acetate, toluene, C₁˜C₁₀ alcohol, dichloromethane, tetrahydrofuran, acetonitrile, isopropyl acetate, dimethylformamide, dimethylacetamide, 2-methyltetrahydrofuran, and the like. A mixture of (R)-5-bromo-2,2-dimethyl-2,3-dihydro-1H-inden-1-amine of Formula 4a and(S)-5-bromo-2,2-dimethyl-2,3-dihydro-1H-inden-1-amine of Formula 4b is obtained, preferably in a weight ratio of 7:3 to 6:4, from the reaction between the mixture of the compound of Formula 6a and the compound of Formula 6b and the acid. The obtained mixture may be isolated according to conventional methods such as neutralizing, extracting, concentrating, and the like; or be used in the subsequent reactions without isolating the residue obtained by the concentration. The reaction between the mixture of the compound of Formula 4a and the compound of Formula 4b and N-acetyl-D-leucine may be carried out in the same manner as in Step (i).

The reaction of Step (t) may be carried out in the same manner as in Step (ii).

For example, the processes for preparing a stereoisomer of the compound of Formula 1 or a pharmaceutically acceptable salt thereof may be shown in Reaction Scheme 2 or Reaction Scheme 3 below.

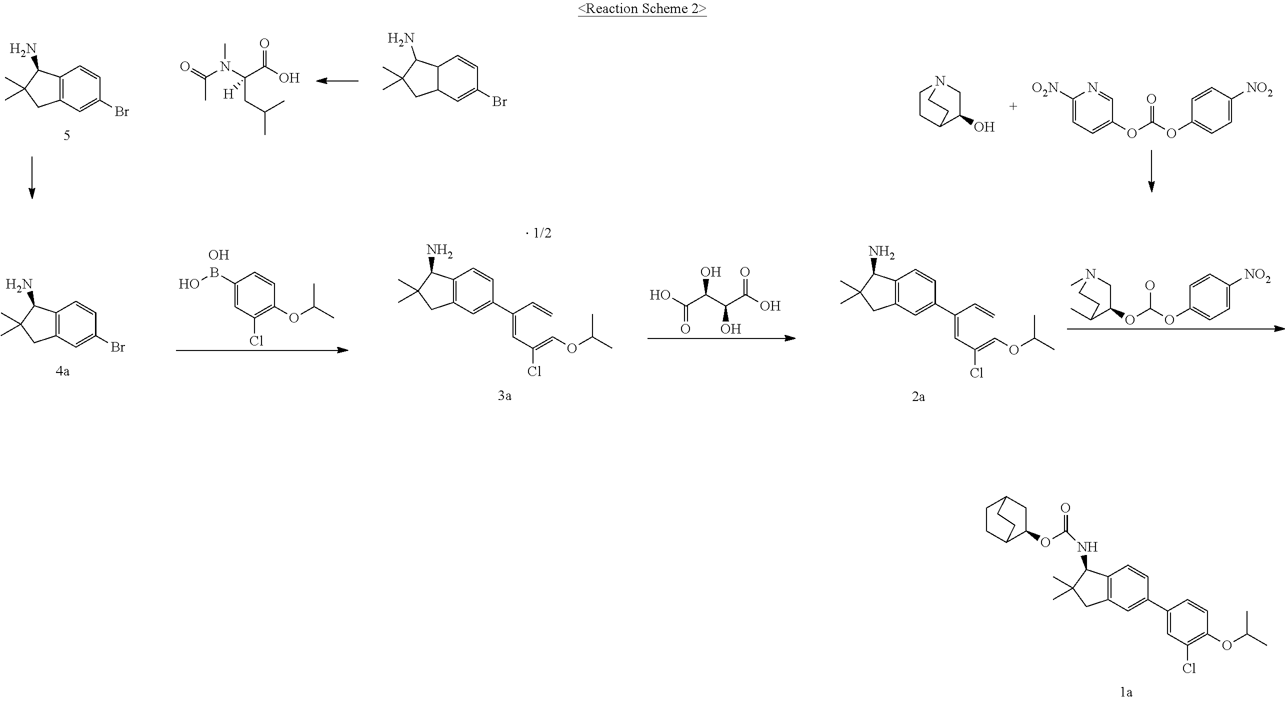

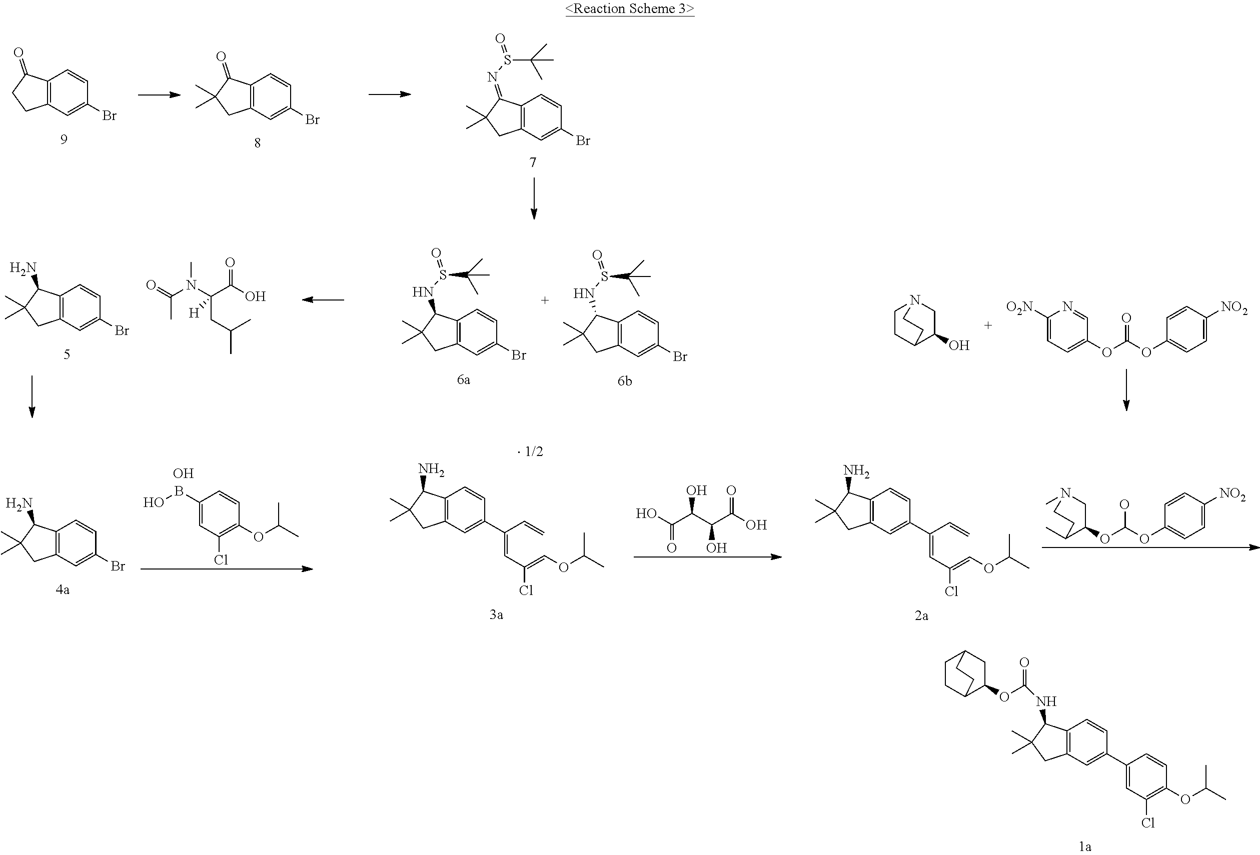

The processes of the present invention comprise reacting a compound of Formula 4 or a compound of Formula 4a with 3-chloro-4-isopropoxyphenylboronic acid, followed by reacting with D-tartaric acid, to prepare a compound of Formula 3 or a compound of Formula 3a [Step (a) or Step (a′)].

The reaction between the compound of Formula 4 or the compound of Formula 4a and 3-chloro-4-isopropoxyphenylboronic acid may be carried out through a Suzuki reaction. That is, the reaction between the compound of Formula 4 or the compound of Formula 4a and 3-chloro-4-isopropoxyphenylboronic acid may be carried out in the presence of a metal catalyst and a base. And, the reaction between the compound of Formula 4 or the compound of Formula 4a and 3-chloro-4-isopropoxyphenylboronic acid may be carried out in the presence of a ligand, in addition to said metal catalyst and base.

The metal catalyst may be one or more selected from the group consisting of palladium, copper, iron, cadmium, zinc, and nickel; may be preferably one or more selected from the group consisting of tetrakis(triphenylphosphine)palladium(0) (Pd(PPh₃)₄), palladium(II) acetate (Pd(OAc)₂), bis(triphenylphosphine)palladium(II) dichloride (PdCl₂(PPh₃)₂), [1,1′-bis(diphenylphosphino)ferrocene]dichloropalladium(II) (Pd(dppf)Cl₂), and tris(dibenzylideneacetone)dipalladium(0) (Pd₂(dba)₃).

The base may be selected from the group consisting of cesium carbonate (Cs₂CO₃), sodium carbonate (Na₂CO₃), potassium carbonate (K₂CO₃), potassium phosphate (K₃PO₄), triethylamine, diisopropylamine, potassium hydroxide, potassium acetate, potassium t-butoxide, sodium hydroxide, sodium hydride, sodium phosphate, 1,8-diazabicyclo[5.4.0]undec-7-ene, 1,4-diazabicyclo[2.2.2]octane, 1,5-diazabicyclo[4.3.0]non-5-ene, pyridine, and diisopropylethylamine.

The ligand may be one or more selected from the group consisting of 2-dicyclohexylphosphino-2′,4′,6′-triisopropylbiphenyl (XPhos), 4,5-bis(diphenylphosphino)-9,9-dimethylxanthene (Xantphos), 2,2′-bis(diphenylphosphino)-1,1′-binaphthyl (BINAP), and 1,1′-bis(diphenylphosphino)ferrocene (DPPF).

The reaction between the compound of Formula 4 or the compound of Formula 4a and 3-chloro-4-isopropoxyphenylboronic acid may be carried out in the presence of one or more solvents selected from the group consisting of water, C₁˜C₁₀ alcohol, dichloromethane, tetrahydrofuran, acetone, acetonitrile, isopropyl acetate, dimethylformamide, dimethylacetamide, toluene, 2-methyltetrahydrofuran, ethyl acetate, and 1,4-dioxane. In an embodiment, the reaction may be carried out in a mixed solvent of water and an organic solvent such as C₁˜C₁₀ alcohol, dichloromethane, tetrahydrofuran, acetone, acetonitrile, toluene, 2-methyltetrahydrofuran, and the like. The volume ratio of the organic solvent and water in the mixed solvent may be in the range of 1:1 to 20:1, but is not limited thereto. The reaction may be carried out in the range of 50° C. to the reflux temperature of the solvent used. Other reaction conditions, including the amount of the palladium catalyst, ligand, and base used, may be determined according to known methods for the Suzuki reaction.

The product obtained according to the Suzuki reaction is crystallized in the form of a compound of Formula 3 or a compound of Formula 3a by the reaction with D-tartaric acid. The compound of Formula 3 or the compound of Formula 3a is suitable for mass production and easy for the scale-up thereof, due to its excellent filterability. The D-tartaric acid may be used in a ratio of 0.4 to 2.0 equivalents based on 1 equivalent of the compound of Formula 4 or the compound of Formula 4a. The reaction may be carried out at 0° C. to 70° C. (e.g., about 50° C.) for 0 to 3 hours (e.g., about 30 minutes) in a mixed solvent of water and an organic solvent such as acetone, C₁˜C₁₀ alcohol, dichloromethane, tetrahydrofuran, acetonitrile, isopropyl acetate, toluene, 2-methyltetrahydrofuran, ethyl acetate, and the like or in an organic solvent such as acetone, and the like. The volume ratio of the organic solvent and water in the mixed solvent may range from 5:1 to 30:1, but is not limited thereto. The product, i.e., the compound of Formula 3 or the compound of Formula 3a, may be isolated accordion to conventional methods such as filtration, drying (e.g., vacuum drying), and the like.

The processes of the present invention comprise reacting the compound of Formula 3 or the compound of Formula 3a with a base to prepare a compound of Formula 2 or a compound of Formula 2a [Step (b) or Step (b′)].

The base used in the step (b) or the step (b′) may be one or more selected from the group consisting of sodium hydroxide, potassium hydroxide, sodium carbonate, sodium bicarbonate, potassium carbonate, potassium phosphate, and sodium phosphate. The base may be used in a ratio of 1 to 5 equivalents based on 1 equivalent of the compound of Formula 3 or the compound of Formula 3a, but is not limited thereto. The reaction between the compound of Formula 3 or the compound of Formula 3a and a base may be carried out in a mixed solvent of water and an organic solvent such as isopropyl acetate, ethyl acetate, dichloromethane, toluene, 2-methyltetrahydrofuran, and the like. The volume ratio of the organic solvent and water in the mixed solvent may be in the range of 1:1 to 10:1, but is not limited thereto. The product, i.e., the compound of Formula 2 or the compound of Formula 2a, may be isolated according to conventional methods such as washing, concentrating (e.g., concentrating under reduced pressure) and the like.

The processes of the present invention comprise reacting the compound of Formula 2 or the compound of Formula 2a with (S)-4-nitrophenyl quinuclidin-3-yl carbonate to prepare a compound of Formula 1 or a compound of Formula 1a [Step (c) or Step (c′)].

The (S)-4-nitrophenyl quinuclidin-3-yl carbonate may be prepared according to a known method (e.g., WO 2021/096238). For example, the (S)-4-nitrophenyl quinuclidin-3-yl carbonate may be obtained by reacting (S)-(+)-3-quinuclidinol with bis(4-nitrophenyl) carbonate in the presence of a base or in the absence of a base. When the reaction is carried out in the presence of a base, the base may be one or more bases selected from the group consisting of sodium carbonate, sodium bicarbonate, potassium carbonate, potassium phosphate, sodium phosphate, 1,8-diazabicyclo[5.4.0]undec-7-ene, 1,4-diazabicyclo[2.2.2]octane, 1,5-diazabicyclo[4.3.0]non-5-ene, pyridine, triethylamine, diisopropylamine, and diisopropylethylamine. The base may be used in a ratio of 1 to 3 equivalents based on 1 equivalent of (S)-(+)-3-quinuclidinol, but is not limited thereto.

It is found by the present invention that the reaction between(S)-(+)-3-quinuclidinol and bis(4-nitrophenyl) carbonate; and the reaction between the compound of Formula 2 or the compound of Formula 2a and(S)-4-nitrophenyl quinuclidin-3-yl carbonate can be carried out in a one-pot reaction. Therefore, the reaction between (S)-(+)-3-quinuclidinol and bis(4-nitrophenyl) carbonate; and the reaction between the compound of Formula 2 or the compound of Formula 2a and(S)-4-nitrophenyl quinuclidin-3-yl carbonate may be preferably carried out in a one-pot reaction.

The reaction between the compound of Formula 2 or the compound of Formula 2a and(S)-4-nitrophenyl quinuclidin-3-yl carbonate may be carried out in the presence of a base or in the absence of a base. When the reaction is carried out in the presence of a base, the base may be one or more bases selected from the group consisting of sodium carbonate, sodium bicarbonate, potassium carbonate, potassium phosphate, sodium phosphate, 1,8-diazabicyclo[5.4.0]undec-7-ene, 1,4-diazabicyclo[2.2.2]octane, 1,5-diazabicyclo[4.3.0]non-5-ene, pyridine, triethylamine, diisopropylamine, and diisopropylethylamine. The base may be used in a ratio of 1 to 3 equivalents based on 1 equivalent of the compound of Formula 2 or the compound of Formula 2a, but is not limited thereto. And, the reaction between the compound of Formula 2 or the compound of Formula 2a and(S)-4-nitrophenyl quinuclidin-3-yl carbonate may be carried out in one or more solvents selected from the group consisting of dimethylacetamide, dimethylformamide, dichloromethane, tetrahydrofuran, acetonitrile, isopropyl acetate, 2-methyltetrahydrofuran, and ethyl acetate. If necessary, the obtained compound of Formula 1 or Formula 1a may be isolated in the form of a pharmaceutically acceptable salt, for example, in the form of a camphorsulfonic acid salt.

The present invention, within its scope, includes a process for preparing a compound of Formula 4a, which is used as a starting material in the preparation of a compound of Formula 1a or a pharmaceutically acceptable salt thereof.

In an embodiment, the present invention includes a process for preparing a compound of Formula 4a comprising:
(i) reacting a compound of Formula 4 with N-acetyl-D-leucine, in a water-miscible organic solvent or a mixed solvent of water and a water-miscible organic solvent, to obtain a compound of Formula 5; and

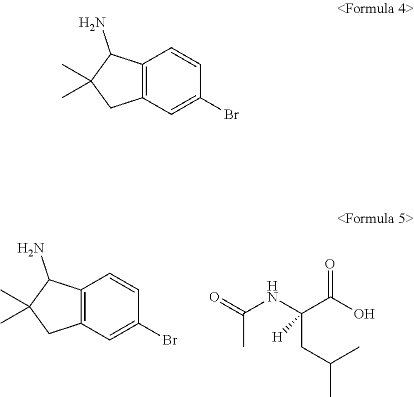

(ii) reacting the compound of Formula 5 with a base to obtain a compound of Formula 4a.

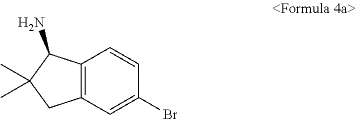

The reactions of Step (i) and Step (ii) may be carried out as described in the above.

In another embodiment, the present invention includes a process for preparing a compound of Formula 4a comprising:
(p) reacting a compound of Formula 9 with iodomethane to prepare a compound of Formula 8;

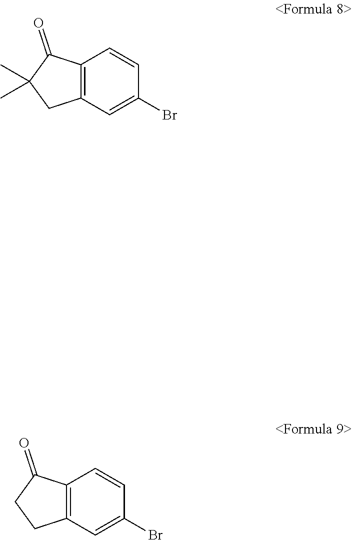

(q) reacting the compound of Formula 8 with (S)-(−)-methyl-2-propanesulfinamide to prepare a compound of Formula 7;

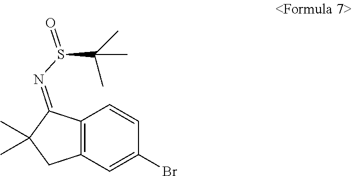 [SMILES: CC1(C)Cc2cc(Br)ccc2/C1=N\[S@@](=O)C(C)(C)C]

(r) reducing the compound of Formula 7 to prepare a mixture of a compound of Formula 6a and a compound of Formula 6b;

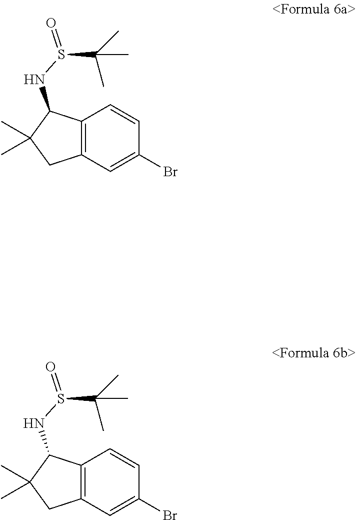 [SMILES: CC1(C)Cc2cc(Br)ccc2[C@@H]1N[S@@](=O)C(C)(C)C ; CC1(C)Cc2cc(Br)ccc2[C@H]1N[S@@](=O)C(C)(C)C]

(s) reacting the mixture of a compound of Formula 6a and a compound of Formula 6b with an acid to prepare a mixture of a compound of Formula 4a and a compound of Formula 4b, reacting the mixture of a compound of Formula 4a and a compound of Formula 4b with N-acetyl-D-leucine in a water-miscible organic solvent or a mixed solvent of water and water-miscible organic solvent, and then isolating a compound of Formula 5; and

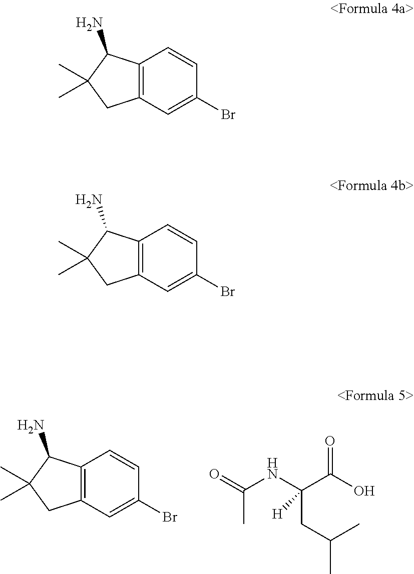

(t) reacting the compound of Formula 5 with a base to obtain a compound of Formula 4a.

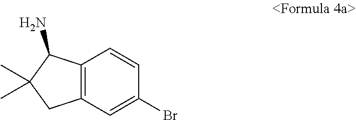

The reactions of Step (p) to Step (t) may be carried out as described in the above.

And, the present invention, within its scope, includes novel intermediates useful in the preparation of a compound of Formula 1 or 1a or a pharmaceutically acceptable salt thereof.

In an embodiment, the present invention includes a compound of Formula 2 useful in the preparation of a compound of Formula 1 or a pharmaceutically acceptable salt thereof:

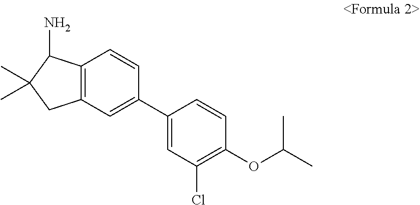 [SMILES: CC(C)Oc1ccc(-c2ccc3c(c2)CC(C)(C)C3N)cc1Cl]

In another embodiment, the present invention includes a compound of Formula 2a useful in the preparation of a compound of Formula 1a or a pharmaceutically acceptable salt thereof:

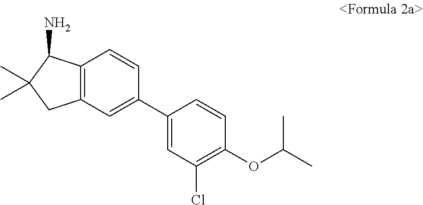 [SMILES: CC(C)Oc1ccc(-c2ccc3c(c2)CC(C)(C)[C@H]3N)cc1Cl]

In still another embodiment, the present invention includes a compound of Formula 3 useful in the preparation of a compound of Formula 1 or a pharmaceutically acceptable salt thereof:

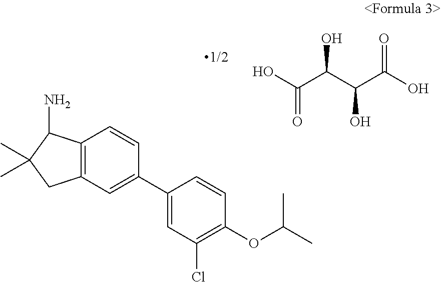 [SMILES: CC(C)Oc1ccc(-c2ccc3c(c2)CC(C)(C)C3N)cc1Cl ; O=C(O)[C@@H](O)[C@H](O)C(=O)O]

In still another embodiment, the present invention includes a compound of Formula 3a useful in the preparation of a compound of Formula 1a or a pharmaceutically acceptable salt thereof:

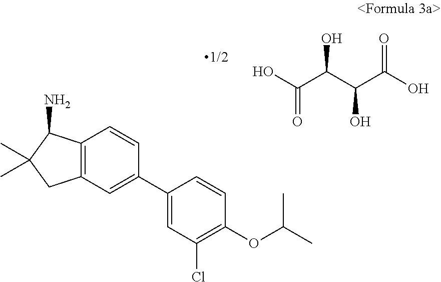 [SMILES: CC(C)Oc1ccc(-c2ccc3c(c2)CC(C)(C)[C@H]3N)cc1Cl ; O=C(O)[C@@H](O)[C@H](O)C(=O)O]

In still another embodiment, the present invention includes a compound of Formula 5 useful in the preparation of a compound of Formula 1a:

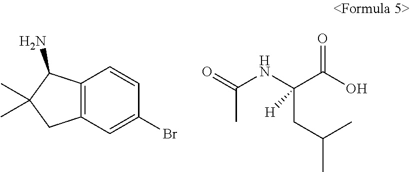

Hereinafter, the present invention will be described in more detail through Examples and Test Examples. However, these Examples and Test Examples are provided for illustration purposes only, and are not intended to limit the scope of the invention.

### Example 1: (R)-5-bromo-2,2-dimethyl-2,3-dihydro-1H-inden-1-amine acetyl-D-leucinate (Compound 5)

After 5-bromo-2,2-dimethyl-2,3-dihydro-1H-inden-1-amine (44 g, 183.2 mmol) was dissolved in a mixed solvent of acetone (792 mL) and purified water (88 mL) at room temperature, N-acetyl-D-leucine (14.3 g, 82.4 mmol) was added thereto. The reaction mixture was stirred at 35° C. for about 19 hours. The resulting solid was filtered and then dried in vacuo to obtain 24.0 g of the titled compound. (Yield: 31.7%, Optical purity: 99.5%)

### Example 2: (R)-5-bromo-2,2-dimethyl-2,3-dihydro-1H-inden-1-amine acetyl-D-leucinate (Compound 5)

After 5-bromo-2,2-dimethyl-2,3-dihydro-1H-inden-1-amine (3 g, 12.5 mmol) was dissolved in a mixed solvent of acetonitrile (54 mL) and purified water (6 mL) at room temperature, N-acetyl-D-leucine (1.0 g, 5.6 mmol) was added thereto. The reaction mixture was stirred at 25° C. for about 20 hours. The resulting solid was filtered and then dried in vacuo to obtain 1.9 g of the titled compound. (Yield: 36.8%, Optical purity: 93.0%)

### Example 3: (R)-5-bromo-2,2-dimethyl-2,3-dihydro-1H-inden-1-amine (Compound 4a)

While a mixture of (R)-5-bromo-2,2-dimethyl-2,3-dihydro-1H-inden-1-amine acetyl-D-leucinate (44 g, 0.106 mol), isopropyl acetate (220 mL), and purified water (220 mL) was stirred at room temperature, 50% sodium hydroxide (14.5 g, 0.181 mol) was added thereto. The reaction mixture was stirred for 20 minutes and then subject to layer separation. The aqueous layer was extracted with isopropyl acetate (132 mL). The combined organic layer was washed with purified water (88 mL), dried over magnesium sulfate (22 g), and then concentrated under reduced pressure to obtain 25.5 g of the titled compound. (Yield: 100%, Optical purity: 99.5%)

### Example 4: 5-bromo-2,2-dimethyl-2,3-dihydro-1H-inden-1-one (Compound 8)

A solution of 85% potassium hydroxide (3.9 g, 59.2 mmol) in purified water (5 mL) was cooled to 0-5° C. A mixture of 5-bromo-2,3-dihydro-1H-inden-1-one (5 g, 23.7 mmol), N-methyl-2-pyrrolidone (30 mL), and iodomethane (7.1 g, 49.8 mmol) was added to the potassium hydroxide solution, while maintaining the temperature below 30° C. The reaction mixture was stirred at room temperature for about 4 hours. Isopropyl acetate (20 mL) and toluene (10 mL) were added to the reaction mixture, which was then stirred. After the organic layer was separated, the resulting aqueous layer was extracted with isopropyl acetate (30 mL). The combined organic layer was washed with purified water (30 mL) and then concentrated to obtain 4.8 g of the titled compound. (Yield: 84.7%)

¹H-NMR (400 MHz, CDCl₃) δ 7.64 (d, 2H), 7.28 (d, 1H), 2.99 (s, 2H), 1.25 (s, 6H)

### Example 5: 5-bromo-2,2-dimethyl-2,3-dihydro-1H-inden-1-one (Compound 8)

A solution of 85% potassium hydroxide (23.5 g, 355.4 mmol) in purified water (30 mL) was cooled to 0-5° C. A mixture of 5-bromo-2,3-dihydro-1H-inden-1-one (30 g, 142.1 mmol), N-methyl-2-pyrrolidone (180 mL), and iodomethane (44.4 g, 312.8 mmol) was added to the potassium hydroxide solution, while maintaining the temperature below 30° C. The reaction mixture was stirred at room temperature for about 4 hours. After purified water (450 mL) was slowly added to the reaction mixture, toluene (300 mL) was added thereto. The reaction mixture was stirred and then filtered with a celite pad. After the organic layer was separated from the filtrate, the resulting aqueous layer was extracted with toluene (300 mL). The combined organic layer was washed with a 10% sodium chloride solution and then concentrated to obtain 30.8 g of the titled compound. (Yield: 90.6%)

¹H-NMR (400 MHz, CDCl₃) δ 7.64 (d, 2H), 7.28 (d, 1H), 2.99 (s, 2H), 1.25 (s, 6H)

### Example 6: (S,Z)—N-(5-bromo-2,2-dimethyl-2,3-dihydro-1H-inden-1-ylidene)-2-methylpropane-2-sulfinamide (Compound 7)

A mixture of 5-bromo-2,2-dimethyl-2,3-dihydro-1H-inden-1-one (10 g, 41.8 mmol), titanium (IV) ethoxide (19.1 g 83.6 mmol), and toluene (100 mL) was stirred at room temperature. (S)-(−)-2-Methylpropanesulfinamide (10.1 g, 83.6 mmol) was added to the mixture, which was stirred at 85° C. for about 19 hours under nitrogen gas flow. The reaction mixture was concentrated and the resulting residue was used in the subsequent reaction without further purification.

¹H-NMR (400 MHz, DMSO-d₆) δ 8.2 (s, 1H), 7.7 (s, 1H), 7.6 (d, 1H), 3.0 (s, 2H), 1.2 (s, 9H), 1.0 (s, 6H)

### Example 7: (S,Z)—N-(5-bromo-2,2-dimethyl-2,3-dihydro-1H-inden-1-ylidene)-2-methylpropane-2-sulfinamide (Compound 7)

A mixture of 5-bromo-2,2-dimethyl-2,3-dihydro-1H-inden-1-one (27 g, 112.9 mmol) and titanium (IV) ethoxide (51.5 g, 225.8 mmol) was stirred at room temperature. (S)-(−)-2-Methylpropanesulfinamide (27.4 g, 225.8 mmol) was added to the mixture, which was stirred at 85° C. for 6 hours under nitrogen gas flow. The reaction mixture was concentrated and the resulting residue was used in the subsequent reaction without further purification.

¹H-NMR (400 MHz, DMSO-d₆) δ 8.2 (s, 1H), 7.7 (s, 1H), 7.6 (d, 1H), 3.0 (s, 2H), 1.2 (s, 9H), 1.0 (s, 6H)

### Example 8: A Mixture of (S)—N—((R)-5-bromo-2,2-dimethyl-2,3-dihydro-1H-inden-1-yl)-2-methylpropane-2-sulfinamide (Compound 6a) and (S)—N—((S)-5-bromo-2,2-dimethyl-2,3-dihydro-1H-inden-1-yl)-2-methylpropane-2-sulfinamide (Compound 6b)

A mixture of (S,Z)—N-(5-bromo-2,2-dimethyl-2,3-dihydro-1H-inden-1-ylidene)-2-methylpropane-2-sulfinamide (192.8 g, 563.3 mmol) and tetrahydrofuran (1.35 L) was cooled to −10° C. Sodium borohydride (23.4 g, 619.6 mmol) was added to the reaction mixture, which was then stirred while slowly raising the temperature to room temperature. After completion of the reaction, the reaction mixture was cooled to 0° C. and then a citric acid solution (2.9 L) was slowly added thereto. The reaction mixture was extracted with ethyl acetate (965 mL). The obtained organic layer was washed with a 10% sodium chloride solution (965 mL) and then concentrated. The resulting residue was used in the subsequent reaction without further purification.

The residue was identified as a mixture of the compound of Formula 6a and the compound of Formula 6b. The NMR data measured after separation using silica gel column chromatography (eluent: hexane/ethyl acetate) are as follows.

<Compound of Formula 6a>

¹H-NMR (400 MHz, CDCl₃) δ 7.3 (m, 2H), 7.1 (d, 1H), 4.4 (d, 1H), 3.5 (d, 1H), 2.8 (q, 2H), 1.4 (s, 3H), 1.3 (s, 9H), 0.96 (s, 3H)

<Compound of Formula 6b>

¹H-NMR (400 MHz, CDCl₃) δ 7.5 (d, 1H), 7.3 (m, 2H), 4.4 (d, 1H), 3.3 (d, 1H), 2.8 (q, 2H), 1.3 (s, 9H), 1.2 (s, 3H), 0.90 (s, 3H)

### Example 9: (R)-5-bromo-2,2-dimethyl-2,3-dihydro-1H-inden-1-amine acetyl-D-leucinate (Compound 5)

A mixture of the residue obtained in Example 8 (i.e., the mixture of the compound of Formula 6a and the compound of Formula 6b) (181.4 g, 526.8 mmol) and ethyl acetate (1.8 L) was cooled to 0° C., followed by stirring. Conc. hydrochloric acid (35%) (183 mL) was added to the reaction mixture, which was warmed to room temperature and then stirred for 4 hours. Purified water (907 mL) was added to the reaction mixture, which was cooled to 0° C. and then the pH thereof was adjusted to pH 9-11 with 50% sodium hydroxide. The separated organic layer was washed with a 10% sodium chloride solution (907 mL) and then concentrated under reduced pressure. Acetone (907 mL) was added to the resulting residue, followed by re-concentration. Acetone (2.27 L) and purified water (253 ml) were added to the resulting concentrated residue, followed by stirring at room temperature. N-acetyl-D-leucine (68.4 g, 395.1 mmol) was added to the reaction mixture, which was then stirred at 35° C. for about 16 hours. The resulting solid was filtered and then dried in vacuo to obtain 84.3 g of the titled compound. (Yield: 38.7% (3-step yield from the compound of Formula 8), Optical purity: 99.2%)

¹H-NMR (400 MHz, DMSO-d6) δ 7.9 (d, 1H), 7.4 (d, 2H), 7.3 (d, 1H), 4.1 (q, 1H), 3.9 (s, 1H), 2.7 (q, 2H), 1.8 (s, 3H), 1.6 (m, 1H), 1.5 (m, 2H), 1.2 (s, 3H), 0.9 (d, 3H), 0.8 (d, 6H)

### Example 10: (R)-5-bromo-2,2-dimethyl-2,3-dihydro-1H-inden-1-amine (Compound 4a)

A mixture of (R)-5-bromo-2,2-dimethyl-2,3-dihydro-1H-inden-1-amine acetyl-D-leucinate (7.5 g, 18.1 mmol), isopropyl acetate (37 mL), and purified water (37 mL) was stirred at room temperature. 50% sodium hydroxide (2.5 g, 53.0 mmol) was added thereto under stirring. After the layer separation thereof, the aqueous layer was extracted with isopropyl acetate (23 mL). The combined organic layer was washed with purified water (20 mL), dried over magnesium sulfate (3.8 g), and then concentrated under reduced pressure to obtain 4.5 g of the titled compound. (Yield: 100%, Optical purity: 97.5%)

¹H-NMR (400 MHz, CDCl₃) δ 7.3 (m, 2H), 7.2 (d, 1H), 3.8 (s, 1H), 2.7 (t, 1H), 1.2 (s, 3H), 0.88 (s, 3H)

### Example 11: 5-(3-chloro-4-isopropoxyphenyl)-2,2-dimethyl-2,3-dihydro-1H-inden-1-amine (2S,3S)-2,3-dihydroxysuccinate (Compound 3)

A mixture of 5-bromo-2,2-dimethyl-2,3-dihydro-1H-inden-1-amine (5.0 g, 0.0208 mol), 3-chloro-4-isopropoxyphenylboronic acid (5.1 g, 0.0238 mol), potassium carbonate (5.8 g, 0.042 mol), [1,1′-bis(diphenylphosphino)ferrocene]dichloropalladium(II) (0.3 g, 0.41 mmol), 2-dicyclohexylphosphino-2′,4′,6′-triisopropylbiphenyl (0.4 g, 0.84 mmol), tetrahydrofuran (45 mL), and purified water (10 mL) was refluxed under stirring for 7 hours. The reaction mixture was concentrated under reduced pressure and then extracted with purified water (15 mL), ethyl acetate (35 mL) and dichloromethane (25 mL). The separated organic layer was washed with purified water (25 mL) and then concentrated under reduced pressure. Acetone (45 mL) was added to the resulting concentrated residue and then a solution of D-tartaric acid (1.56 g, 0.0104 mol) in purified water (5 mL) were added thereto. The reaction mixture was heated to 50° C., stirred for 30 minutes, and then cooled to room temperature. The resulting solid was filtered and then dried in vacuo to obtain 7.9 g of the titled compound. (Yield: 93.7%, Purity: 94.8%)

¹H-NMR (400 MHz, DMSO) δ 7.70 (s, 1H), 7.69-7.43 (m, 4H), 7.23 (d, 1H), 4.05 (s, 1H), 3.80 (s, 1H), 2.76 (q, 2H), 1.32 (d, 6H), 1.20 (s, 3H), 0.95 (s, 3H)

### Example 12: (R)-5-(3-chloro-4-isopropoxyphenyl)-2,2-dimethyl-2,3-dihydro-1H-inden-1-amine (2S,3S)-2,3-dihydroxysuccinate (Compound 3a)

A mixture of (R)-5-bromo-2,2-dimethyl-2,3-dihydro-1H-inden-1-amine (3.0 g, 0.0125 mol), 3-chloro-4-isopropoxyphenylboronic acid (3.1 g, 0.0144 mol), potassium carbonate (3.5 g, 0.025 mol), [1,1′-bis(diphenylphosphino)ferrocene]dichloropalladium(II) (0.18 g, 0.25 mmol), 2-dicyclohexylphosphino-2′,4′,6′-triisopropylbiphenyl (0.24 g, 0.5 mmol), tetrahydrofuran (27.0 mL), and purified water (6 mL) was stirred at 60° C. for 4 hours. Purified water (15 mL) was added to the reaction mixture, which was concentrated under reduced pressure and then extracted with ethyl acetate (15 mL). The obtained extract was washed with purified water (15 mL) and then concentrated under reduced pressure. Acetone (27 mL) was added to the resulting concentrated residue and then D-tartaric acid (0.94 g, 0.006 mol) and purified water (3 mL) were added thereto. The reaction mixture was heated to 50° C., stirred for 30 minutes, and then cooled to room temperature. The resulting solid was filtered and then dried in vacuo to obtain 4.4 g of the titled compound. (Yield: 87.0%)

¹H-NMR (400 MHz, MeOD) δ 7.61 (s, 1H), 7.59-7.50 (d, 1H), 7.49-7.47 (d, 3H), 7.14-7.12 (d, 2H), 4.70-4.64 (m, 1H), 4.35 (d, 2H), 3.0 (d, 1H), 2.82 (d, 1H), 1.39 (d, 6H), 1.27-1.23 (d, 6H)

### Example 13: (R)-5-(3-chloro-4-isopropoxyphenyl)-2,2-dimethyl-2,3-dihydro-1H-inden-1-amine (2S,3S)-2,3-dihydroxysuccinate (Compound 3a)

A mixture of (R)-5-bromo-2,2-dimethyl-2,3-dihydro-1H-inden-1-amine (5.0 g, 0.0208 mol), 3-chloro-4-isopropoxyphenylboronic acid (5.13 g, 0.0239 mol), potassium carbonate (5.76 g, 0.0416 mol), bis(triphenylphosphinc)palladium(II) dichloride (0.3 g, 0.416 mmol), 2-dicyclohexylphosphino-2′,4′,6′-triisopropylbiphenyl (0.4 g, 0.833 mmol), acetone (35 mL), and purified water (5 mL) was stirred at 60° C. for 4 hours. Purified water (25 mL) was added to the reaction mixture, which was concentrated under reduced pressure and then extracted with ethyl acetate (15 mL). The obtained extract was washed with purified water (25 mL) and then concentrated under reduced pressure. Acetone (45 mL) was added to the resulting concentrated residue and then D-tartaric acid (1.56 g, 10.41 mmol) and purified water (5 mL) were added thereto. The reaction mixture was heated to 50° C., stirred for 30 minutes, and then cooled to room temperature. The resulting solid was filtered and then dried in vacuo to obtain 6.7 g of the titled compound. (Yield: 79.5%)

¹H-NMR (400 MHz, MeOD) δ 7.61 (s, 1H), 7.59-7.50 (d, 1H), 7.49-7.47 (d, 3H), 7.14-7.12 (d, 2H), 4.70-4.64 (m, 1H), 4.35 (d, 2H), 3.0 (d, 1H), 2.82 (d, 1H), 1.39 (d, 6H), 1.27-1.23 (d, 6H)

### Example 14: (R)-5-(3-chloro-4-isopropoxyphenyl)-2,2-dimethyl-2,3-dihydro-1H-inden-1-amine (2S,3S)-2,3-dihydroxysuccinate (Compound 3a)

A mixture of (R)-5-bromo-2,2-dimethyl-2,3-dihydro-1H-inden-1-amine (50.0 g, 0.208 mol), 3-chloro-4-isopropoxyphenylboronic acid (43.8 g, 0.204 mol), potassium carbonate (57.6 g, 0.416 mol), bis(triphenylphosphine)palladium(II) dichloride (3.0 g, 4.164 mmol), ethanol (225 mL), and purified water (150 mL) was refluxed under stirring for 2 hours. The reaction mixture was concentrated under reduced pressure and then methyl tert-butyl ether (500 mL) and a 3N hydrochloric acid solution (500 mL) were slowly added thereto. To the separated organic layer, was added purified water (250 mL). The reaction mixture was neutralized with a 50% sodium hydroxide solution. The separated organic layer was washed with purified water and then concentrated under reduced pressure. Acetone (250 mL) was added to the resulting concentrated residue and then concentrated under reduced pressure. Acetone (450 mL) was added again to the resulting concentrated residue and then a solution of D-tartaric acid (15.6 g, 0.104 mol) in acetone (250 mL) were added thereto. The reaction mixture was refluxed under stirring for 30 minutes, and then cooled to room temperature. The resulting solid was filtered and then dried in vacuo to obtain 71.3 g of the titled compound. (Yield: 84.6%)

¹H-NMR (400 MHz, MeOD) δ 7.61 (s, 1H), 7.59-7.50 (d, 1H), 7.49-7.47 (d, 3H), 7.14-7.12 (d, 2H), 4.70-4.64 (m, 1H), 4.35 (d, 2H), 3.0 (d, 1H), 2.82 (d, 1H), 1.39 (d, 6H), 1.27-1.23 (d, 6H)

### Example 15: (R)-5-(3-chloro-4-isopropoxyphenyl)-2,2-dimethyl-2,3-dihydro-1H-inden-1-amine (2S,3S)-2,3-dihydroxysuccinate (Compound 3a)

A mixture of (R)-5-bromo-2,2-dimethyl-2,3-dihydro-1H-inden-1-amine (10.0 g, 0.0416 mol), 3-chloro-4-isopropoxyphenylboronic acid (10.3 g, 0.0479 mol), potassium carbonate (11.51 g, 0.0833 mol), [1,1′-bis(diphenylphosphino)ferrocene]dichloropalladium(II) (0.61 g, 0.0008 mol), 2-dicyclohexylphosphino-2′,4′,6′-triisopropylbiphenyl (0.79 g, 0.002 mol), tetrahydrofuran (90.0 mL), and purified water (20 mL) was refluxed under stirring for 3 hours. Purified water (50 mL) was added to the reaction mixture, which was concentrated under reduced pressure and then extracted with ethyl acetate (50 mL). The obtained extract was washed with purified water (50 mL) and then concentrated under reduced pressure. Acetone (90 mL) was added to the resulting concentrated residue and then D-tartaric acid (3.13 g, 0.021 mol) and purified water (10 mL) were added thereto. The reaction mixture was heated to 50° C., stirred for 30 minutes, and then cooled to room temperature. The resulting solid was filtered and then dried in vacuo to obtain 13.95 g of the titled compound. (Yield: 82.7%)

¹H-NMR (400 MHz, MeOD) δ 7.61 (s, 1H), 7.59-7.5 (d, 1H), 7.49-7.47 (d, 3H), 7.14-7.12 (d, 2H), 4.70-4.64 (m, 1H), 4.35 (d, 2H), 3.0 (d, 1H), 2.82 (d, 1H), 1.39 (d, 6H), 1.27-1.23 (d, 6H)

### Example 16: (R)-5-(3-chloro-4-isopropoxyphenyl)-2,2-dimethyl-2,3-dihydro-1H-inden-1-amine (2S,3S)-2,3-dihydroxysuccinate (Compound 3a)

A mixture of (R)-5-bromo-2,2-dimethyl-2,3-dihydro-1H-inden-1-amine (3.0 g, 0.0125 mol), 3-chloro-4-isopropoxyphenylboronic acid (3.1 g, 0.0144 mol), potassium carbonate (3.5 g, 0.025 mol), [1,1′-bis(diphenylphosphino)ferrocene]dichloropalladium(II) (0.18 g, 0.003 mol), 2-dicyclohexylphosphino-2′,4′,6′-triisopropylbiphenyl (0.24 g, 0.005 mol), tetrahydrofuran (27.0 mL), and purified water (4.8 mL) was refluxed under stirring for 4 hours. Purified water (15 mL) was added to the reaction mixture, which was concentrated under reduced pressure and then extracted with ethyl acetate (15 mL). The obtained extract was washed with purified water (15 mL) and then concentrated under reduced pressure. Acetone (27 mL) was added to the resulting concentrated residue and then D-tartaric acid (0.94 g, 0.006 mol) and purified water (3 mL) were added thereto. The reaction mixture was heated to 50° C., stirred for 30 minutes, and then cooled to room temperature. The resulting solid was filtered and then dried in vacuo to obtain 4.3 g of the titled compound. (Yield: 85.0%)

¹H-NMR (400 MHz, MeOD) δ 7.61 (s, 1H), 7.59-7.5 (d, 1H), 7.49-7.47 (d, 3H), 7.14-7.12 (d, 2H), 4.70-4.64 (m, 1H), 4.35 (d, 2H), 3.0 (d, 1H), 2.82 (d, 1H), 1.39 (d, 6H), 1.27-1.23 (d, 6H)

### Example 17: (R)-5-(3-chloro-4-isopropoxyphenyl)-2,2-dimethyl-2,3-dihydro-1H-inden-1-amine (2S,3S)-2,3-dihydroxysuccinate (Compound 3a)

A mixture of (R)-5-bromo-2,2-dimethyl-2,3-dihydro-1H-inden-1-amine (3.0 g, 0.0125 mol), 3-chloro-4-isopropoxyphenylboronic acid (3.1 g, 0.0144 mol), potassium carbonate (3.5 g, 0.025 mol), [1,1′-bis(diphenylphosphino)ferrocene]dichloropalladium(II) (0.18 g, 0.003 mol), 2-dicyclohexylphosphino-2′,4′,6′-triisopropylbiphenyl (0.24 g, 0.005 mol), tetrahydrofuran (27.0 mL), and purified water (3.9 mL) was refluxed under stirring for 4 hours. Purified water (15 mL) was added to the reaction mixture, which was concentrated under reduced pressure and then extracted with ethyl acetate (15 mL). The obtained extract was washed with purified water (15 mL) and then concentrated under reduced pressure. Acetone (27 mL) was added to the resulting concentrated residue and then D-tartaric acid (0.94 g, 0.006 mol) and purified water (3 mL) were added thereto. The reaction mixture was heated to 50° C., stirred for 30 minutes, and then cooled to room temperature. The resulting solid was filtered and then dried in vacuo to obtain 4.3 g of the titled compound. (Yield: 85.0%)

¹H-NMR (400 MHz, MeOD) δ 7.61 (s, 1H), 7.59-7.5 (d, 1H), 7.49-7.47 (d, 3H), 7.14-7.12 (d, 2H), 4.70-4.64 (m, 1H), 4.35 (d, 2H), 3.0 (d, 1H), 2.82 (d, 1H), 1.39 (d, 6H), 1.27-1.23 (d, 6H)

### Example 18: (R)-5-(3-chloro-4-isopropoxyphenyl)-2,2-dimethyl-2,3-dihydro-1H-inden-1-amine (2S,3S)-2,3-dihydroxysuccinate (Compound 3a)

A mixture of (R)-5-bromo-2,2-dimethyl-2,3-dihydro-1H-inden-1-amine (3.0 g, 0.0125 mol), 3-chloro-4-isopropoxyphenylboronic acid (3.1 g, 0.0144 mol), potassium carbonate (3.5 g, 0.025 mol), [1,1′-bis(diphenylphosphino)ferrocene]dichloropalladium(II) (0.18 g, 0.003 mol), 2-dicyclohexylphosphino-2′,4′,6′-triisopropylbiphenyl (0.24 g, 0.005 mol), tetrahydrofuran (27.0 mL), and purified water (3.0 mL) was refluxed under stirring for 2 hours. Purified water (15 mL) was added to the reaction mixture, which was concentrated under reduced pressure and then extracted with ethyl acetate (15 mL). The obtained extract was washed with purified water (15 mL) and then concentrated under reduced pressure. Acetone (27 mL) was added to the resulting concentrated residue and then D-tartaric acid (0.94 g, 0.006 mol) and purified water (3 mL) were added thereto. The reaction mixture was heated to 50° C., stirred for 30 minutes, and then cooled to room temperature. The resulting solid was filtered and then dried in vacuo to obtain 4.2 g of the titled compound. (Yield: 83.0%)

¹H-NMR (400 MHz, MeOD) δ 7.61 (s, 1H), 7.59-7.5 (d, 1H), 7.49-7.47 (d, 3H), 7.14-7.12 (d, 2H), 4.70-4.64 (m, 1H), 4.35 (d, 2H), 3.0 (d, 1H), 2.82 (d, 1H), 1.39 (d, 6H), 1.27-1.23 (d, 6H)

### Example 19: (R)-5-(3-chloro-4-isopropoxyphenyl)-2,2-dimethyl-2,3-dihydro-1H-inden-1-amine (2S,3S)-2,3-dihydroxysuccinate (Compound 3a)

A mixture of (R)-5-bromo-2,2-dimethyl-2,3-dihydro-1H-inden-1-amine (3.0 g, 0.0125 mol), 3-chloro-4-isopropoxyphenylboronic acid (3.1 g, 0.0144 mol), potassium carbonate (3.5 g, 0.025 mol), [1,1′-bis(diphenylphosphino)ferrocene]dichloropalladium(II) (0.18 g, 0.003 mol), 2-dicyclohexylphosphino-2′,4′,6′-triisopropylbiphenyl (0.24 g, 0.005 mol), tetrahydrofuran (27.0 mL), and purified water (2.1 mL) was refluxed under stirring for 2 hours. Purified water (15 mL) was added to the reaction mixture, which was concentrated under reduced pressure and then extracted with ethyl acetate (15 mL). The obtained extract was washed with purified water (15 mL) and then concentrated under reduced pressure. Acetone (27 mL) was added to the resulting concentrated residue and then D-tartaric acid (0.94 g, 0.006 mol) and purified water (3 mL) were added thereto. The reaction mixture was heated to 50° C., stirred for 30 minutes, and then cooled to room temperature. The resulting solid was filtered and then dried in vacuo to obtain 4.4 g of the titled compound. (Yield: 87.0%)

¹H-NMR (400 MHz, MeOD) δ 7.61 (s, 1H), 7.59-7.5 (d, 1H), 7.49-7.47 (d, 3H), 7.14-7.12 (d, 2H), 4.70-4.64 (m, 1H), 4.35 (d, 2H), 3.0 (d, 1H), 2.82 (d, 1H), 1.39 (d, 6H), 1.27-1.23 (d, 6H)

### Example 20: (R)-5-(3-chloro-4-isopropoxyphenyl)-2,2-dimethyl-2,3-dihydro-1H-inden-1-amine (2S,3S)-2,3-dihydroxysuccinate (Compound 3a)

A mixture of (R)-5-bromo-2,2-dimethyl-2,3-dihydro-1H-inden-1-amine (10.0 g, 0.0416 mol), 3-chloro-4-isopropoxyphenylboronic acid (10.3 g, 0.0479 mol), potassium carbonate (11.51 g, 0.0833 mol), [1,1′-bis(diphenylphosphino)ferrocene]dichloropalladium(II) (0.61 g, 0.0008 mol), 2-dicyclohexylphosphino-2′,4′,6′-triisopropylbiphenyl (0.79 g, 0.002 mol), tetrahydrofuran (90.0 mL), and purified water (20 mL) was stirred at 55° C. for 4 hours. Purified water (50 mL) was added to the reaction mixture, which was concentrated under reduced pressure and then extracted with ethyl acetate (50 mL). The obtained extract was washed with purified water (50 mL) and then concentrated under reduced pressure. Acetone (90 mL) was added to the resulting concentrated residue and then D-tartaric acid (3.13 g, 0.021 mol) and purified water (10 mL) were added thereto. The reaction mixture was heated to 50° C., stirred for 30 minutes, and then cooled to room temperature. The resulting solid was filtered and then dried in vacuo to obtain 13.95 g of the titled compound. (Yield: 82.7%)

¹H-NMR (400 MHz, MeOD) δ 7.61 (s, 1H), 7.59-7.5 (d, 1H), 7.49-7.47 (d, 3H), 7.14-7.12 (d, 2H), 4.70-4.64 (m, 1H), 4.35 (d, 2H), 3.0 (d, 1H), 2.82 (d, 1H), 1.39 (d, 6H), 1.27-1.23 (d, 6H)

### Example 21: (R)-5-(3-chloro-4-isopropoxyphenyl)-2,2-dimethyl-2,3-dihydro-1H-inden-1-amine (2S,3S)-2,3-dihydroxysuccinate (Compound 3a)

A mixture of (R)-5-bromo-2,2-dimethyl-2,3-dihydro-1H-inden-1-amine (3.0 g, 0.0125 mol), 3-chloro-4-isopropoxyphenylboronic acid (3.1 g, 0.0144 mol), potassium carbonate (3.5 g, 0.025 mol), [1,1′-bis(diphenylphosphino)ferrocene]dichloropalladium(II) (0.18 g, 0.003 mol), 2-dicyclohexylphosphino-2′,4′,6′-triisopropylbiphenyl (0.24 g, 0.005 mol), acetone (27.0 mL), and purified water (3 mL) was refluxed under stirring for 7 hours. Purified water (15 mL) was added to the reaction mixture, which was concentrated under reduced pressure and then extracted with ethyl acetate (15 mL). The obtained extract was washed with purified water (15 mL) and then concentrated under reduced pressure. Acetone (27 mL) was added to the resulting concentrated residue and then D-tartaric acid (0.94 g, 0.006 mol) and purified water (3 mL) were added thereto. The reaction mixture was heated to 50° C., stirred for 30 minutes, and then cooled to room temperature. The resulting solid was filtered and then dried in vacuo to obtain 4.1 g of the titled compound. (Yield: 81.1%)

¹H-NMR (400 MHz, MeOD) δ 7.61 (s, 1H), 7.59-7.5 (d, 1H), 7.49-7.47 (d, 3H), 7.14-7.12 (d, 2H), 4.70-4.64 (m, 1H), 4.35 (d, 2H), 3.0 (d, 1H), 2.82 (d, 1H), 1.39 (d, 6H), 1.27-1.23 (d, 6H)

### Example 22: (R)-5-(3-chloro-4-isopropoxyphenyl)-2,2-dimethyl-2,3-dihydro-1H-inden-1-amine (2S,3S)-2,3-dihydroxysuccinate (Compound 3a)

A mixture of (R)-5-bromo-2,2-dimethyl-2,3-dihydro-1H-inden-1-amine (5.0 g, 0.0208 mol), 3-chloro-4-isopropoxyphenylboronic acid (5.13 g, 0.0239 mol), potassium carbonate (5.76 g, 0.0416 mol), [1,1′-bis(diphenylphosphino)ferrocene]dichloropalladium(II) (0.29 g, 0.0004 mol), acetone (27.0 mL), and purified water (6 mL) was refluxed under stirring for 24 hours. Purified water (25 mL) was added to the reaction mixture, which was concentrated under reduced pressure and then extracted with ethyl acetate (25 mL). The obtained extract was washed with purified water (25 mL) and then concentrated under reduced pressure. Acetone (45 mL) was added to the resulting concentrated residue and then D-tartaric acid (1.56 g, 0.0104 mol) and purified water (3 mL) were added thereto. The reaction mixture was heated to 50° C., stirred for 30 minutes, and then cooled to room temperature. The resulting solid was filtered and then dried in vacuo to obtain 7.0 g of the titled compound. (Yield: 83.0%)

¹H-NMR (400 MHz, MeOD) δ 7.61 (s, 1H), 7.59-7.5 (d, 1H), 7.49-7.47 (d, 3H), 7.14-7.12 (d, 2H), 4.70-4.64 (m, 1H), 4.35 (d, 2H), 3.0 (d, 1H), 2.82 (d, 1H), 1.39 (d, 6H), 1.27-1.23 (d, 6H)

### Example 23: 5-(3-chloro-4-isopropoxyphenyl)-2,2-dimethyl-2,3-dihydro-1H-inden-1-amine (Compound 2)

50% Sodium hydroxide (1.7 g, 0.022 mol) was added under stirring to a mixture of 5-(3-chloro-4-isopropoxyphenyl)-2,2-dimethyl-2,3-dihydro-1H-inden-1-amine (2S,3S)-2,3-dihydroxysuccinate (7.3 g, 0.018 mol), dichloromethane (73 mL), and purified water (51 mL). The reaction mixture was stirred at room temperature for about 30 minutes and then subject to layer separation. The organic layer was washed with purified water (37 mL) and then concentrated under reduced pressure to obtain 6.0 g of the titled compound. (Yield: 100.0%)

¹H NMR (400 MHz, CDCl₃) 7.60 (s, 1H), 7.41-7.33 (m, 4H), 7.00 (d, 1H), 4.59 (m, 1H), 3.94 (s, 1H), 2.74 (s, 2H), 1.41 (d, 6H), 1.24 (s, 3H), 0.91 (s, 3H)

### Example 24: (R)-5-(3-chloro-4-isopropoxyphenyl)-2,2-dimethyl-2,3-dihydro-1H-inden-1-amine (Compound 2a)

50% Sodium hydroxide (4.8 g, 0.059 mol) was added under stirring to a mixture of (R)-5-(3-chloro-4-isopropoxyphenyl)-2,2-dimethyl-2,3-dihydro-1H-inden-1-amine (2S,3S)-2,3-dihydroxysuccinate (20.0 g, 0.049 mol), dichloromethane (200 mL), and purified water (140 mL). The reaction mixture was stirred at room temperature for about 30 minutes and then subject to layer separation. The organic layer was washed with purified water (75 mL) and then concentrated under reduced pressure to obtain 16.3 g of the titled compound. (Yield: 100.0%)

¹H NMR (400 MHz, CDCl₃) 7.60 (s, 1H), 7.41-7.33 (m, 4H), 7.00 (d, 1H), 4.59 (m, 1H), 3.94 (s, 1H), 2.74 (s, 2H), 1.41 (d, 6H), 1.24 (s, 3H), 0.91 (s, 3H)

### Example 25: (S)-quinuclidin-3-yl ((R)-5-(3-chloro-4-isopropoxyphenyl)-2,2-dimethyl-2,3-dihydro-1H-inden-1-yl)carbamate (Compound 1a)

A mixture of (S)-3-quinuclidinol (50.9 g, 0.400 mol), tetrahydrofuran (1320 mL), and bis(4-nitrophenyl) carbonate (126.8 g, 0.417 mol) was stirred at 40-45° C. for 21 hours. To the reaction mixture, were added (R)-5-(3-chloro-4-isopropoxyphenyl)-2,2-dimethyl-2,3-dihydro-1H-inden-1-amine (110.0 g, 0.335 mol) and tetrahydrofuran (330 mL). The reaction mixture was refluxed under stirring for about 15 hours and then cooled to room temperature. Purified water (880 mL) was added to the reaction mixture and the reaction mixture was cooled to 15° C. 50% sodium hydroxide (66.7 g, 0.834 mol) was added to the reaction mixture, which was concentrated under reduced pressure. To the resulting residue, were added purified water (1320 mL) and isopropyl acetate (1650 mL). The separated organic layer was washed with a 2N sodium hydroxide solution (1100 mL) and purified water (550 mL), and then concentrated under reduced pressure to obtain 148.5 g of the titled compound. (Yield: 92.2%)

¹H-NMR (400 MHz, CDCl₃) δ 7.59 (s, 1H), 7.41 (m, 2H), 7.34 (s, 1H), 7.29 (m, 1H), 7.00 (d, 1H), 4.98 (d, 1H), 4.84-4.67 (m, 2H), 4.58 (m, 1H), 3.30 (m, 1H), 2.90-2.73 (m, 7H), 2.09 (m, 1H), 1.85 (m, 1H), 1.70 (m, 1H), 1.60 (m, 1H), 1.42 (m, 1H), 1.41 (d, 6H), 1.30 (s, 3H), 0.98 (s, 3H)

### Example 26: (S)-quinuclidin-3-yl ((R)-5-(3-chloro-4-isopropoxyphenyl)-2,2-dimethyl-2,3-dihydro-1H-inden-1-yl)carbamate (Compound 1a)

A mixture of (S)-3-quinuclidinol (1.39 g, 10.91 mmol), acetonitrile (36 mL), and bis(4-nitrophenyl) carbonate (3.32 g, 10.91 mmol) was stirred at 40° C. for 6 hours. To the reaction mixture, were added (R)-5-(3-chloro-4-isopropoxyphenyl)-2,2-dimethyl-2,3-dihydro-1H-inden-1-amine (3.0 g, 9.09 mmol) and acetonitrile (9 mL). The reaction mixture was stirred at 70° C. for about 15 hours and then cooled to room temperature. Purified water (24 mL) and 50% sodium hydroxide were added to the reaction mixture, which was then stirred at room temperature for about 1 hour. Purified water (12 mL) was added to the reaction mixture, which was then concentrated under reduced pressure. To the resulting residue, was added ethyl acetate (51 mL). The separated organic layer was washed with a 5% sodium bicarbonate solution, dried over magnesium sulfate, and then concentrated under reduced pressure to obtain 3.8 g of the titled compound. (Yield: 86.5%)

¹H-NMR (400 MHz, CDCl₃) δ 7.59 (s, 1H), 7.41 (m, 2H), 7.34 (s, 1H), 7.29 (m, 1H), 7.00 (d, 1H), 4.98 (d, 1H), 4.84-4.67 (m, 2H), 4.58 (m, 1H), 3.30 (m, 1H), 2.90-2.73 (m, 7H), 2.09 (m, 1H), 1.85 (m, 1H), 1.70 (m, 1H), 1.60 (m, 1H), 1.42 (m, 1H), 1.41 (d, 6H), 1.30 (s, 3H), 0.98 (s, 3H)

### Example 27: (S)-quinuclidin-3-yl ((R)-5-(3-chloro-4-isopropoxyphenyl)-2,2-dimethyl-2,3-dihydro-1H-inden-1-yl)carbamate (Compound 1a)

A mixture of (S)-3-quinuclidinol (4.6 g, 0.036 mol), acetonitrile (120 mL), diisopropylcthylamine (7.8 g, 0.06 mol), and bis(4-nitrophenyl) carbonate (11.5 g, 0.038 mol) was stirred at 40° C. for 8 hours. To the reaction mixture, were added (R)-5-(3-chloro-4-isopropoxyphenyl)-2,2-dimethyl-2,3-dihydro-1H-inden-1-amine (10.0 g, 0.03 mol) and acetonitrile (30 mL). The reaction mixture was refluxed under stirring for about 15 hours and then cooled to room temperature. Purified water (80 mL) was added to the reaction mixture, which was then concentrated under reduced pressure. 50% sodium hydroxide (6.1 g, 0.076 mol) was added to the reaction mixture, which was stirred at room temperature for about 1 hour and then concentrated under reduced pressure. To the resulting residue, were added purified water (120 mL) and isopropyl acetate (150 mL). The separated organic layer was washed with a 2N sodium hydroxide solution (100 mL), washed with purified water (50 mL), and then concentrated under reduced pressure to obtain 13.0 g of the titled compound. (Yield: 88.8%)

¹H-NMR (400 MHz, CDCl₃) δ 7.59 (s, 1H), 7.41 (m, 2H), 7.34 (s, 1H), 7.29 (m, 1H), 7.00 (d, 1H), 4.98 (d, 1H), 4.84-4.67 (m, 2H), 4.58 (m, 1H), 3.30 (m, 1H), 2.90-2.73 (m, 7H), 2.09 (m, 1H), 1.85 (m, 1H), 1.70 (m, 1H), 1.60 (m, 1H), 1.42 (m, 1H), 1.41 (d, 6H), 1.30 (s, 3H), 0.98 (s, 3H)

### Example 28: ((1S,4R)-7,7-dimethyl-2-oxobicyclo[2.2.1]heptan-1-yl) methanesulfonic acid salt of (S)-quinuclidin-3-yl (5-(3-chloro-4-isopropoxyphenyl)-2,2-dimethyl-2,3-dihydro-1H-inden-1-yl) carba mate ((1S)-(+)-10-camphorsulfonic acid salt of Compound 1)

A mixture of (S)-3-quinuclidinol (2.8 g, 0.022 mol), tetrahydrofuran (72 mL), and bis(4-nitrophenyl) carbonate (6.9 g, 0.023 mol) was stirred at 40-45° C. for 2 hours. To the reaction mixture, were added 5-(3-chloro-4-isopropoxyphenyl)-2,2-dimethyl-2,3-dihydro-1H-inden-1-amine (6.0 g, 0.018 mol) and tetrahydrofuran (18 mL). The reaction mixture was refluxed under stirring for about 15 hours and then cooled to room temperature. Purified water (48 mL) was added to the reaction mixture. 50% sodium hydroxide (3.6 g, 0.045 mol) was added to the reaction mixture, which was then concentrated under reduced pressure. To the resulting residue, were added purified water (72 mL) and isopropyl acetate (105 mL). The separated organic layer was washed with a 2N sodium hydroxide solution (60 mL) and purified water (30 mL) sequentially and then concentrated under reduced pressure. The resulting residue was dissolved in methyl ethyl ketone (78 mL). The resulting solution was heated to 50° C. and then (1S)-(+)-10-camphorsulfonic acid (4.2 g, 0.018 mol) was added thereto. The reaction mixture was cooled to about 25° C. and then stirred for about 1 hour. After the reaction mixture was filtered under reduced pressure, the resulting wet cake was washed with methyl ethyl ketone (18 mL). The resulting solid was dried in vacuo to obtain 4.0 g of the titled compound. (Yield: 30.7%, Purity: 93.6%)

¹H-NMR (400 MHz, DMSO-d₆) δ 9.47 (s, 1H), 7.68-7.67 (d, 1H), 7.64-7.62 (d, 1H), 7.57-7.54 (m, 1H), 7.46-7.44 (m, 2H), 7.27-7.22 (m, 2H), 4.93-4.90 (m, 1H), 4.82-4.66 (m, 2H), 3.73-3.68 (m, 1H), 3.28-3.19 (m, 5H), 2.89-2.85 (d, 1H), 2.80-2.64 (m, 3H), 2.39-2.36 (d, 1H), 2.28-2.20 (m, 2H), 2.08-2.03 (m, 1H), 1.95-1.73 (m, 6H), 1.33-1.31 (d, 6H), 1.30-1.26 (m, 2H), 1.16 (s, 3H), 1.05 (s, 3H), 0.90 (s, 3H), 0.74 (s, 3H)

### Example 29: ((1S,4R)-7,7-dimethyl-2-oxobicyclo[2.2.1]heptan-1-yl) methanesulfonic acid salt of (S)-quinuclidin-3-yl ((R)-5-(3-chloro-4-isopropoxyphenyl)-2,2-dimethyl-2,3-dihydro-1H-inden-1-yl)carbamate ((1S)-(+)-10-camphorsulfonic Acid Salt of Compound 1a)

After(S)-quinuclidin-3-yl ((R)-5-(3-chloro-4-isopropoxyphenyl)-2,2-dimethyl-2,3-dihydro-1H-inden-1-yl) carba mate (148.5 g, 0.307 mol) was dissolved in methyl ethyl ketone (1430 mL) and the reaction mixture was heated to 50° C., (1S)-(+)-10-camphorsulfonic acid (77.5 g, 0.334 mol) dissolved in purified water (33 mL) was added thereto. The reaction mixture was cooled to about 25° C., and then stirred for about 1 hour. After the reaction mixture was filtered under reduced pressure, the resulting wet cake was washed with methyl ethyl ketone (330 mL). The resulting solid was dried in vacuo to obtain 178.0 g of the titled compound. (Yield: 80.9%, Optical purity: 99.5%)

¹H-NMR (400 MHz, DMSO-d₆) δ 9.47 (s, 1H), 7.68-7.67 (d, 1H), 7.64-7.62 (d, 1H), 7.57-7.54 (m, 1H), 7.46-7.44 (m, 2H), 7.27-7.22 (m, 2H), 4.93-4.90 (m, 1H), 4.82-4.66 (m, 2H), 3.73-3.68 (m, 1H), 3.28-3.19 (m, 5H), 2.89-2.85 (d, 1H), 2.80-2.64 (m, 3H), 2.39-2.36 (d, 1H), 2.28-2.20 (m, 2H), 2.08-2.03 (m, 1H), 1.95-1.73 (m, 6H), 1.33-1.31 (d, 6H), 1.30-1.26 (m, 2H), 1.16 (s, 3H), 1.05 (s, 3H), 0.90 (s, 3H), 0.74 (s, 3H)

### Comparative Example: (S)-quinuclidin-3-yl ((R)-5-(3-chloro-4-isopropoxyphenyl)-2,2-dimethyl-2,3-dihydro-1H-inden-1-yl)carbamate (Compound 1a)

A mixture of (S)-quinuclidin-3-yl ((R)-5-bromo-2,2-dimethyl-2,3-dihydro-1H-inden-1-yl) carbamate (20.0 g, 0.050 mol), cesium carbonate (33.1 g, 0.102 mol), 3-chloro-4-isopropoxyphenylboronic acid (12.0 g, 0.0056 mol), [1,1′-bis(diphenylphosphino)ferrocene]dichloropalladium(II) (1.86 g, 0.0025 mol), 2-dicyclohexylphosphino-2′,4′,6′-triisopropylbiphenyl (2.42 g, 0.0051 mol), tetrahydrofuran (180 mL), and purified water (20 mL) was stirred at 60° C. for 5 hours. The reaction mixture was concentrated under reduced pressure and then purified water (100 mL) and ethyl acetate (200 mL) were added thereto. Purified water (100 mL) was added to the separated organic layer and then the pH thereof was adjusted to pH 1-2 with conc. hydrochloric acid. The separated organic layer was concentrated under reduced pressure to obtain 20.0 g of the titled compound. (Yield: 81.4%)

¹H-NMR (400 MHz, CDCl₃) δ 7.59 (s, 1H), 7.41 (m, 2H), 7.34 (s, 1H), 7.29 (m, 1H), 7.00 (d, 1H), 4.98 (d, 1H), 4.84-4.67 (m, 2H), 4.58 (m, 1H), 3.30 (m, 1H), 2.90-2.73 (m, 7H), 2.09 (m, 1H), 1.85 (m, 1H), 1.70 (m, 1H), 1.60 (m, 1H), 1.42 (m, 1H), 1.41 (d, 6H), 1.30 (s, 3H), 0.98 (s, 3H)

### Test Example: Measurements of the Residual Amount of Palladium

The residual amounts of palladium in the products prepared in Comparative Example, Example 25, and Example 29 were measured. Specifically, we used an ORS (Octopole Reaction system) which reduces the interferences through the collisions to ionized atoms by injected helium gas. Agilent's 7800 ICP-MS model was used as the instrument. A calibration curve was prepared using the multi-element stock solution (Agilent) and each residual amount of palladium was measured by quantitative analysis of the sample pretreated with a diluted hydrochloric acid solution. The results are shown in Table 1 below.

TABLE 1

| | Residual amount of Pd | |
|---|---|---|
| Comparative Example | 1417 | ppm |
| Example 25 | 380 | ppm |
| Example 29 | 9 | ppm |

As can be seen from the results of Table 1, when Compound 1 is prepared according to the present invention, the residual amount of palladium in the product can be remarkably reduced.

1.-38. (canceled)

39. A process for preparing a compound of Formula 1 or a pharmaceutically acceptable salt thereof, the process comprising:
(a) reacting a compound of Formula 4 with 3-chloro-4-isopropoxyphenylboronic acid, followed by reacting with D-tartaric acid, to prepare a compound of Formula 3;
(b) reacting the compound of Formula 3 with a base to prepare a compound of Formula 2; and
(c) reacting the compound of Formula 2 with (S)-4-nitrophenyl quinuclidin-3-yl carbonate to prepare a compound of Formula 1.

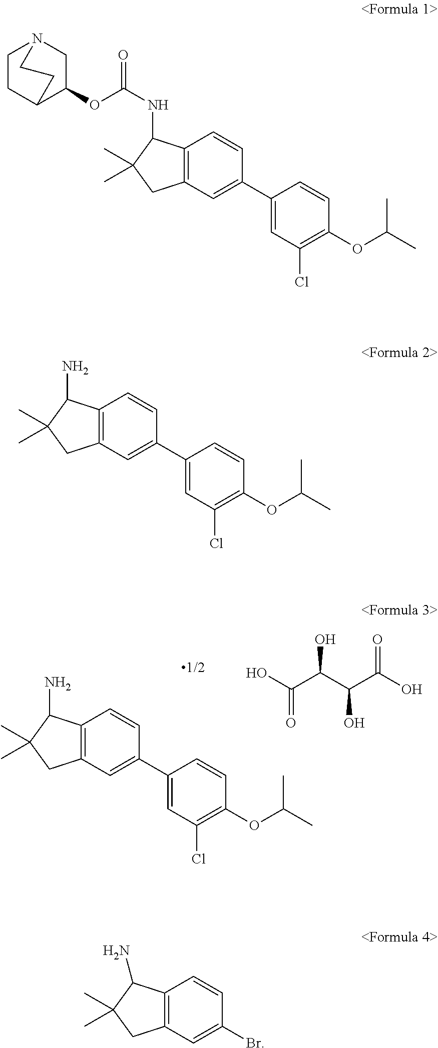

40. A process for preparing a compound of Formula 1a or a pharmaceutically acceptable salt thereof, the process comprising:
(a′) reacting a compound of Formula 4a with 3-chloro-4-isopropoxyphenylboronic acid, followed by reacting with D-tartaric acid, to prepare a compound of Formula 3a;
(b′) reacting the compound of Formula 3a with a base to prepare a compound of Formula 2a; and
(c′) reacting the compound of Formula 2a with (S)-4-nitrophenyl quinuclidin-3-yl carbonate to prepare a compound of Formula 1a.

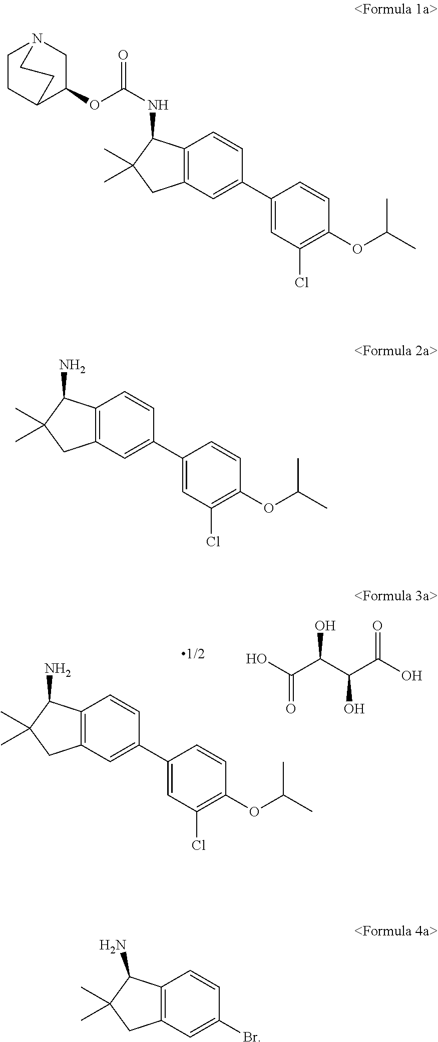

41. The process as claimed in claim 40, wherein the compound of Formula 4a is obtained by a process comprising:
(i) reacting a compound of Formula 4 with N-acetyl-D-leucine, in a water-miscible organic solvent or a mixed solvent of water and a water-miscible organic solvent, to obtain a compound of Formula 5; and 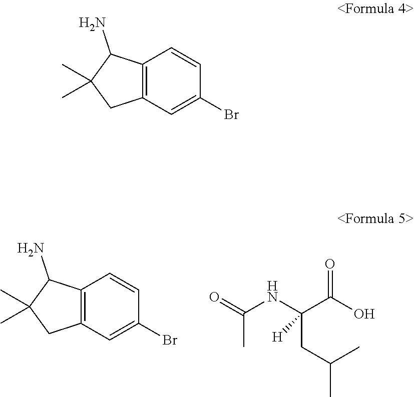
(ii) reacting the compound of Formula 5 with a base to obtain a compound of Formula 4a.

42. The process as claimed in claim 41, wherein the water-miscible organic solvent is one or more selected from the group consisting of acetone, acetonitrile, C₁˜C₅ alcohol, tetrahydrofuran, methyl ethyl ketone, isopropyl acetate, ethyl acetate, toluene, methyl tert-butyl ether, 2-methyltetrahydrofuran, and dichloromethane.

43. The process as claimed in claim 41, wherein the base is one or more selected from the group consisting of sodium hydroxide, potassium hydroxide, potassium carbonate, sodium carbonate, calcium hydroxide, sodium bicarbonate, and potassium phosphate.

44. The process as claimed in claim 40, wherein the compound of Formula 4a is obtained by a process comprising:
(p) reacting a compound of Formula 9 with iodomethane to prepare a compound of Formula 8; 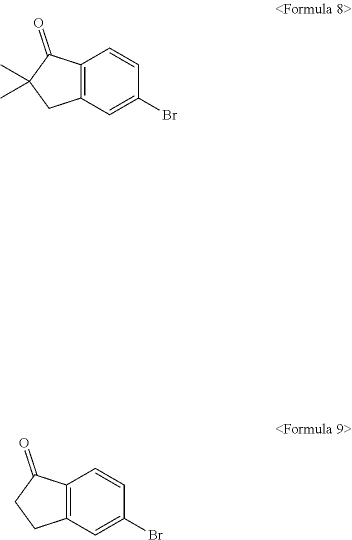
(q) reacting the compound of Formula 8 with (S)-(−)-methyl-2-propanesulfinamide to prepare a compound of Formula 7; 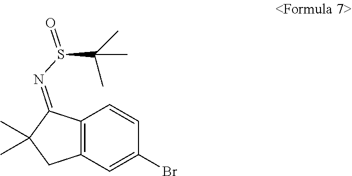 [SMILES: CC1(C)Cc2cc(Br)ccc2/C1=N\[S@@](=O)C(C)(C)C]
(r) reducing the compound of Formula 7 to prepare a mixture of a compound of Formula 6a and a compound of Formula 6b; 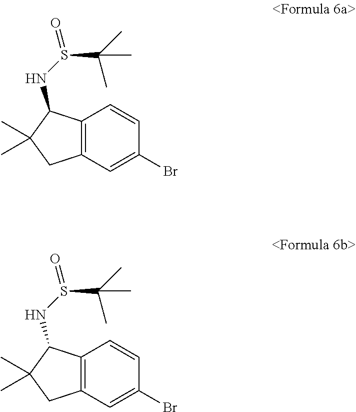 [SMILES: CC1(C)Cc2cc(Br)ccc2[C@@H]1N[S@@](=O)C(C)(C)C ; CC1(C)Cc2cc(Br)ccc2[C@H]1N[S@@](=O)C(C)(C)C]
(s) reacting the mixture of a compound of Formula 6a and a compound of Formula 6b with an acid to prepare a mixture of a compound of Formula 4a and a compound of Formula 4b, reacting the mixture of a compound of Formula 4a and a compound of Formula 4b with N-acetyl-D-leucine in a water-miscible organic solvent or a mixed solvent of water and water-miscible organic solvent, and then isolating a compound of Formula 5; and 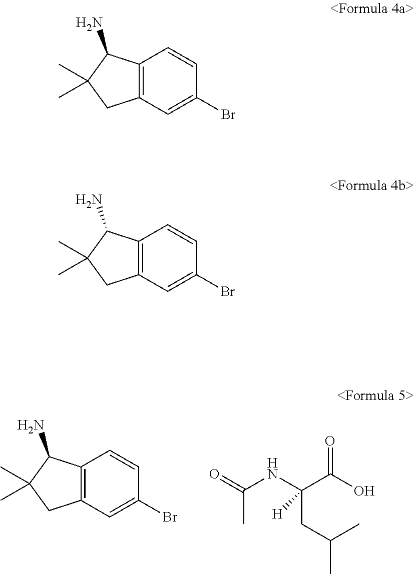
(t) reacting the compound of Formula 5 with a base to obtain a compound of Formula 4a.

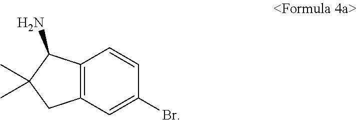

45. The process as claimed in claim 44, wherein the reaction of the step (p) is carried out in the presence of a base selected from the group consisting of cesium carbonate (CS₂CO₃), sodium carbonate (Na₂CO₃), potassium carbonate (K₂CO₃), potassium phosphate (K₃PO₄), triethylamine, diisopropylamine, potassium hydroxide, potassium acetate, potassium t-butoxide, sodium hydroxide, sodium hydride, sodium phosphate, 1,8-diazabicyclo[5.4.0]undec-7-ene, 1,4-diazabicyclo[2.2.2]octane, 1,5-diazabicyclo[4.3.0]non-5-ene, pyridine, and diisopropylethylamine.

46. The process as claimed in claim 44, wherein the reaction of the step (q) is carried out in the presence of a titanium catalyst selected from the group consisting of titanium ethoxide and titanium isopropoxide.

47. The process as claimed in claim 44, wherein the step (r) is carried out by using one or more reducing agents selected from the group consisting of sodium triacetoxyborohydride, sodium cyanoborohydride, and sodium borohydride.

48. The process as claimed in claim 44, wherein the acid in the step(s) is selected from the group consisting of hydrochloric acid, sulfuric acid, and nitric acid.

49. The process as claimed in claim 44, wherein the water-miscible organic solvent in the step(s) is one or more selected from the group consisting of acetone, acetonitrile, C₁˜C₅ alcohol, tetrahydrofuran, methyl ethyl ketone, isopropyl acetate, ethyl acetate, toluene, methyl tert-butyl ether, 2-methyltetrahydrofuran, and dichloromethane.

50. The process as claimed in claim 39, wherein the reacting a compound of Formula 4 with 3-chloro-4-isopropoxyphenylboronic acid is carried out in the presence of a metal catalyst selected from the group consisting of palladium, copper, iron, cadmium, zinc, and nickel and a base selected from the group consisting of cesium carbonate (Cs₂CO₃), sodium carbonate (Na₂CO₃), potassium carbonate (K₂CO₃), potassium phosphate (K₃PO₄), triethylamine, diisopropylamine, potassium hydroxide, potassium acetate, potassium t-butoxide, sodium hydroxide, sodium hydride, sodium phosphate, 1,8-diazabicyclo[5.4.0]undec-7-ene, 1,4-diazabicyclo[2.2.2]octane, 1,5-diazabicyclo[4.3.0]non-5-ene, pyridine, and diisopropylethylamine.

51. The process as claimed in claim 40, wherein the reacting a compound of Formula 4a with 3-chloro-4-isopropoxyphenylboronic acid is carried out in the presence of a metal catalyst selected from the group consisting of palladium, copper, iron, cadmium, zinc, and nickel and a base selected from the group consisting of cesium carbonate (Cs₂CO₃), sodium carbonate (Na₂CO₃), potassium carbonate (K₂CO₃), potassium phosphate (K₃PO₄), triethylamine, diisopropylamine, potassium hydroxide, potassium acetate, potassium t-butoxide, sodium hydroxide, sodium hydride, sodium phosphate, 1,8-diazabicyclo[5.4.0]undec-7-ene, 1,4-diazabicyclo[2.2.2]octane, 1,5-diazabicyclo[4.3.0]non-5-ene, pyridine, and diisopropylethylamine.

52. The process as claimed in claim 50, wherein the reacting is carried out in the presence of one or more ligands selected from the group consisting of 2-dicyclohexylphosphino-2′,4′,6′-triisopropylbiphenyl, 4,5-bis(diphenylphosphino)-9,9-dimethylxanthene, 2,2′-bis(diphenylphosphino)-1,1′-binaphthyl, and 1,1′-bis(diphenylphosphino)ferrocene.

53. The process as claimed in claim 51, wherein the reacting is carried out in the presence of one or more ligands selected from the group consisting of 2-dicyclohexylphosphino-2′,4′,6′-triisopropylbiphenyl, 4,5-bis(diphenylphosphino)-9,9-dimethylxanthene, 2,2′-bis(diphenylphosphino)-1,1′-binaphthyl, and 1,1′-bis(diphenylphosphino)ferrocene.

54. The process as claimed in claim 39, wherein the (S)-4-nitrophenyl quinuclidin-3-yl carbonate is obtained by reacting (S)-(+)-3-quinuclidinol with bis(4-nitrophenyl) carbonate.

55. The process as claimed in claim 54, wherein the reacting (S)-(+)-3-quinuclidinol with bis(4-nitrophenyl) carbonate and the reacting the compound of Formula 2 with (S)-4-nitrophenyl quinuclidin-3-yl carbonate are carried out in a one-pot reaction.

56. The process as claimed in claim 40, wherein the (S)-4-nitrophenyl quinuclidin-3-yl carbonate is obtained by reacting (S)-(+)-3-quinuclidinol with bis(4-nitrophenyl) carbonate.

57. The process as claimed in claim 56, wherein the reacting (S)-(+)-3-quinuclidinol with bis(4-nitrophenyl) carbonate and the reacting the compound of Formula 2a with (S)-4-nitrophenyl quinuclidin-3-yl carbonate are carried out in a one-pot reaction.

58. A compound selected from the consisting of Formula 2, 2a, 3, 3a, and 5: [chemical structure]
